(19) United States
(12) Patent Application Publication
(10) Pub. No.: US 2012/0101153 A1
(43) Pub. Date: Apr. 26, 2012

(54) ROCAGLAOL DERIVATIVES AS CARDIOPROTECTANT AGENTS AND AS ANTINEOPLASTIC AGENTS

(75) Inventors: Laurent Desaubry, Illkirch (FR); Gunsel Canan Desaubry, Illkirch (FR); Thierry Cresteil, Chatenay Malabry (FR); Gülen Türkeri, Illkirch (FR)

(73) Assignee: Universite De Strasbourg, Strasbourg (FR)

(21) Appl. No.: 13/130,140

(22) PCT Filed: Nov. 24, 2009
(86) PCT No.: PCT/EP2009/065698

§ 371 Date: Dec. 7, 2011

(30) Foreign Application Priority Data
Nov. 25, 2008 (EP) 08305835.4

(51) Int. Cl. A61K 31/343; A61P 9/00; C07D 307/93

(52) U.S. Cl. 514/468; 549/458

(57) ABSTRACT

The present invention discloses new rocaglaol derivatives and the use of rocaglaol derivatives to prevent or to limit the cardiotoxicity of an antineoplastic agent, in particular to prevent or to limit the apoptosis of cardiomyocytes induced by such agent.

### FIELD OF THE INVENTION

The present invention relates to the prevention of antineoplastic agent cardiotoxicity by using new cardioprotectant agents. In addition, the present invention also relates to new antineoplastic agents.

### BACKGROUND OF THE INVENTION

Cardiotoxicity is a major side effect of various antineoplastic agents including, for instance, anthracyclines, cyclophosphamide, fluorouracil, rituximab, cisplatine, gleevec or herceptin (Pai et al., 2000). Among them, anthracyclines are a class of chemotherapeutic agents which is used to treat a wide range of cancers, including, for instance, leukemias, lymphomas, and breast, uterine, ovarian, and lung cancers. Nevertheless, the clinical utility of anthracycline anticancer agents, especially doxorubicin, is limited by a progressive toxic cardiomyopathy linked to mitochondrial damage and cardiomyocyte apoptosis (Swain et al., 2003). This cardiotoxicity often presents as electrocardiogram changes and arrhythmias, or as a cardiomyopathy leading to congestive heart failure, sometime many years after treatment (Steinherz et al., 1995), and is related to a patient's cumulative lifetime dose. Some recent studies have revealed that when the cumulative dose of doxorubicin reaches 700 mg/m², the risks of developing congestive heart failure dramatically increase to 48% (Swain et al., 2003). Then, this dose is calculated during treatment, and anthracycline treatment is usually stopped or re-evaluated upon reaching the maximum cumulative dose of the particular anthracycline. Important researches have been carried out to identify methods or drugs which are able to prevent or limit the cardiotoxicity of anthracyclines (Wounters et al., 2005). Several strategies have been explored (e.g. US 2007-834799, US 2007-711490, WO 2006/063091 or WO 2007/101925), but the use of dexrazoxane (Zinecard®, Cardioxane®) remains the only one with a clinical proven efficacy. Dexrazoxane is then usually used in conjunction with doxorubicin as a cardioprotectant agent to reduce the risk of cardiotoxicity side effects of anthracycline chemotherapy. As a derivative of EDTA, dexrazoxane chelates iron, but the precise mechanism by which it protects the heart is not known.

Nevertheless, the use of dexrazoxane does not provide a full protection against heart damages and some studies revealed that patients treated with it might have a lower anti-tumour response rate to the anthracycline treatment (Van Dalen, et al., 2005; Van Dalen, et al., 2008). Moreover, the effectiveness of dexrazoxane in children is not established yet.

Therefore, there is a strong need for cardioprotectant agents that can efficiently prevent the cardiotoxicity of anthracyclines or other antineoplastic agents without having any deleterious effect on the anti-tumour response. This need is still stronger for children who are more susceptible to cardiotoxicity of these agents.

### SUMMARY OF THE INVENTION

The object of the present invention is to provide new cardioprotectant agents. The inventors have shown that rocaglaol and some of its derivatives are able to prevent or limit apoptosis induced by the cardiotoxicity of antineoplastic agents, without any deleterious effect on the anti-tumor activity of these agents.

In a first aspect, the invention concerns a rocaglaol derivative of the formula (II)

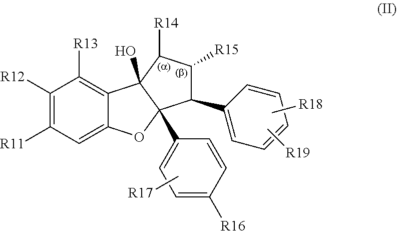

wherein
R¹¹ is alkoxy, optionally substituted;
R¹² is hydrogen;
or R¹¹ and R¹² together form a —O—CH₂—O— unit;
R¹³ is selected from hydrogen and (C₁-C₃)-alkoxy;
R¹⁴ is selected from hydroxyl, oxo group, —OCOR²⁰, R²⁰ being selected from hydrogen and (C₁-C₃)-alkyl, ═N—OR²⁶ with R²⁶ being H or methyl, —NH—(CH₂)ₘ—R²⁷, wherein m is 0 or 1 and R²⁷ is selected from H, —OH, —SO₂Me, —COR²⁸ with R²⁸ being H or (C₁-C₃)-alkyl;
R¹⁵ is selected from hydrogen, —COOR²¹, —CONR²²R²³ and —CONH(CH₂)ₙOH, wherein n is 2, 3 or 4, wherein
   R²¹ and R²³ are independently selected from hydrogen and (C₁-C₃)-alkyl and R²² is selected from the group consisting of (C₁-C₃)-alkoxy, (C₁-C₃)-alkyl and hydrogen, or
   R²² and R²³ together form a 5 atom heterocycle, optionally substituted by a group NHCO—(C₁-C₄)-alkyl;
or R¹⁴, R¹⁵ and carbons (α) and (β) bearing R¹⁴ and R¹⁵ together form

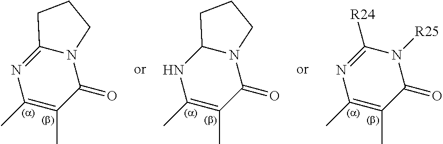

wherein R²⁴ and R²⁵ are independently selected from the group consisting of (C₁-C₃)-alkyl and hydrogen, preferably from hydrogen and methyl;
R¹⁶ is selected from hydrogen, (C₁-C₃)-alkoxy and halogen;
R¹⁷ is in ortho-position to R₁₆ and is selected from hydrogen, (C₁-C₃)-alkoxy and hydroxyl or form together with R¹⁷ a —O—CH₂—O— unit;
R¹⁸ and R¹⁹ are independently selected from the group consisting of hydrogen and halogen, preferably from hydrogen, F, Br and Cl, more preferably are hydrogen or F;
   or any pharmaceutically acceptable salt thereof, for use for preventing or limiting the cardiotoxicity of an antineoplastic agent, preferably an anthracycline, more preferably doxorubicin.

In another aspect, the invention also concerns a method for preventing or limiting the cardiotoxicity of an antineoplastic agent in a subject comprising administering a therapeutically effective amount of a rocaglaol derivative of the formula (II) as described above or any pharmaceutically acceptable salt thereof to said subject. The present invention further concerns the use of a rocaglaol derivative of the formula (II) as described above or any pharmaceutically acceptable salt thereof for preparing a medicament for preventing or limiting the cardiotoxicity of an antineoplastic agent, preferably an anthracycline, more preferably doxorubicin.

Preferably, the rocaglaol derivative of the formula (II) has one or several of the following features:
a) R¹² is H and R¹¹ is a (C₁-C₃)-alkoxy, preferably selected from methoxy and ethoxy, more preferably R¹¹ is methoxy;
b) R¹³ is selected from hydrogen, methoxy and ethoxy, more preferably R¹³ is hydrogen or methoxy;
c) R¹⁴ is selected from hydroxyl, —OCOR²⁰ with R²⁰ being selected from hydrogen, methyl and ethyl, —NH—(CH₂)ₘ—R²⁷, wherein m is 0 and R²⁷ is —SO₂Me or —COR²⁸ with R²⁸ being H or (C₁-C₃)-alkyl, preferably H, methyl or ethyl, more preferably H or methyl;
d) R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂;
e) R¹⁶ is selected from hydrogen, (C₁-C₃)-alkoxy and halogen, more preferably from hydrogen, chlorine, bromine, fluorine and methoxy, still more preferably from bromine and methoxy;
f) R¹⁷ is hydrogen;
g) R¹⁸ is in para and is H or fluorine and R¹⁹ is hydrogen.

In a particular embodiment, the rocaglaol derivative presents the formula (IIa) or (IIb)

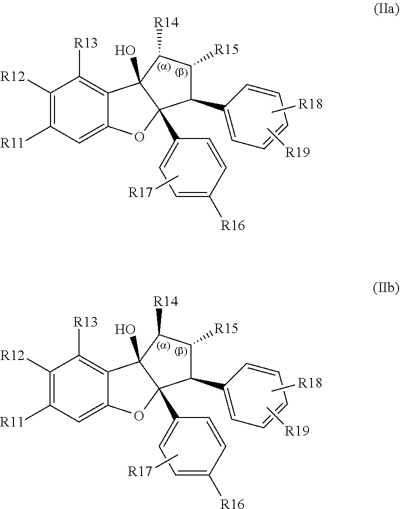

wherein the definition of R11, R12, R13, R14, R15, R16, R17, R18 and R19 is the same as above.

More particularly, the rocaglaol derivative has the formula (IIa) and R¹⁴ is selected from hydroxyl, —OCOR²⁰ with R²⁰ being selected from hydrogen and (C₁-C₃)-alkyl, and ═N—OR²⁶ with R²⁶ being H or methyl. Preferably, R¹⁴ is selected from hydroxyl and —OCOR²⁰ with R²⁰ being selected from hydrogen, methyl and ethyl. More preferably, R¹¹ is alkoxy, especially methoxy or ethoxy, more preferably methoxy; R¹² is hydrogen; R¹³ is selected from hydrogen, methoxy and ethoxy, more preferably R¹³ is hydrogen or methoxy; R¹⁵ is selected from hydrogen, COOMe, CONH₂, CONHMe and CONMe₂; R¹⁶ is selected from the group consisting of hydrogen, chlorine, bromine, fluorine and methoxy, more preferably of bromine and methoxy; R¹⁸ is in para and is H or fluorine; and, R¹⁷ and R¹⁹ are hydrogen. Alternatively, the rocaglaol derivative has the formula (IIb) and R¹⁴ is —NH—(CH₂)ₘ—R²⁷, wherein m is 0 and R²⁷ is —SO₂Me or —COR²⁸ with R²⁸ being H or (C₁-C₃)-alkyl, preferably H or methyl. In particular, R¹¹ is alkoxy, especially methoxy or ethoxy, more preferably methoxy; R¹² is hydrogen; R¹³ is selected from hydrogen, methoxy and ethoxy, more preferably R¹³ is hydrogen or methoxy, still more preferably R¹³ is methoxy; R¹⁵ is selected from hydrogen, COOMe, CONH₂, CONHMe and CONMe₂, more preferably is hydrogen; R¹⁶ is selected from the group consisting of hydrogen, chlorine, bromine, fluorine and methoxy, more preferably is chlorine, bromine, or fluorine, still more preferably is bromine; and, R¹⁷, R¹⁸ and R¹⁹ are hydrogen.

In a particularly preferred embodiment, the rocaglaol derivative is selected from the group consisting of FL1, FL3, FL5, FL6, FL7, FL8, FL9, FL10, FL12, FL13, FL14, FL15, FL16, FL17, FL18, FL19, FL20, FL21, FL22, FL23, FL24 and FL25, more preferably from the group consisting of FL1, FL3, FL5, FL8, FL9, FL10, FL12, FL13, FL14, FL15, FL16, FL17, FL18, FL19, FL20, FL21, FL22, FL23 and FL25.

In a second aspect, the invention provides new rocaglaol derivatives of the formula (I)

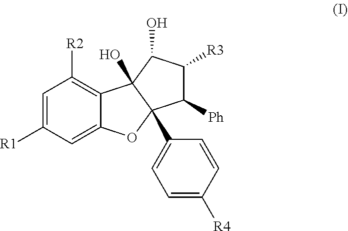

wherein
   R³ is —CONH₂;
   and wherein
      R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine;
      R² is selected from the group consisting of hydrogen and (C₁-C₃)-alkoxy, preferably from hydrogen and methoxy; and,
      R⁴ is halogen, preferably bromine, chloride, iodide, more preferably bromine,
   or,
      R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine;
      R² is hydrogen; and,
      R⁴ is selected from the group consisting of hydrogen and alkoxy,
   or,
      R¹ is substituted alkoxy, preferably a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine;
      R² is (C₁-C₃)-alkoxy, preferably methoxy; and
      R⁴ is selected from the group consisting of hydrogen and alkoxy,
or wherein
   R³ is —COOR⁵ wherein R⁵ is (C₁-C₃)-alkyl, preferably methyl;
   R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine;
   R² is hydrogen; and
   R⁴ is selected from the group consisting of hydrogen, methoxy and halogen;
or wherein
   R¹ is methoxy, and R⁴ is bromine,
   and wherein
      R² is (C₁-C₃)-alkoxy, preferably methoxy, and,
      R³ is hydrogen,
   or
      R² is hydrogen, and,
      R³ is selected from the group consisting of hydrogen, —COOR⁵ and —CONR⁶R⁷, wherein R⁵ is (C₁-C₃)-alkyl, preferably methyl, and R⁶ and R⁷ are independently selected from the group consisting of hydrogen and (C₁-C₃)-alkyl, preferably from hydrogen and methyl;
         or any pharmaceutically acceptable salt thereof.

In a preferred embodiment, R¹ is selected from the group consisting of methoxy; R² is hydrogen; R³ is selected from the group consisting of —COOR⁵ and —CONH₂, wherein R⁵ is methyl; and, R⁴ is halogen or methoxy, preferably bromine or methoxy.

In another preferred embodiment, R¹ is methoxy; R² is selected from the group consisting of methoxy or hydrogen; R³ is —CONH₂; and, R⁴ is halogen, preferably bromine.

In a further preferred embodiment, R¹ and R² are methoxy, R³ is hydrogen and R⁴ is bromine. In an additional preferred embodiment, R¹ is methoxy; R² is hydrogen; R³ is selected from the group consisting of hydrogen, —COO(CH₃) and —CON(CH₃)₂; and, R⁴ is bromine.

In another preferred embodiment, R³ is COOMe, R¹ is methoxy, R² is hydrogen and R⁴ is selected from the group consisting of hydrogen, methoxy and bromine, preferably is methoxy or bromine.

In a further preferred embodiment, R¹ is methoxy, and R⁴ is bromine, and wherein either R² is methoxy and R³ is hydrogen, or R² is hydrogen and R³ is selected in the group consisting of hydrogen, —COO(CH₃), —CONH₂, —CONH(CH₃) and —CON(CH₃)₂.

In particular, the rocaglaol derivative is selected from the group consisting of FL3, FL5, FL6, FL7, FL8, FL9 and FL14.

The present invention further provides a new rocaglaol derivative the formula (II), wherein
R¹¹ is alkoxy, optionally substituted;
R¹² is hydrogen;
or R¹¹ and R¹² together form a —O—CH₂—O-unit;
R¹³ is selected from hydrogen and (C₁-C₃)-alkoxy;
R¹⁴ is —NH—R²⁷, wherein R²⁷ is selected from —SO₂Me, —COR²⁸ with R²⁸ being H or (C₁-C₃)-alkyl;
R¹⁵ is selected from hydrogen, —COOR²¹, —CONR²²R²³ and —CONH(CH₂)ₙOH, wherein n is 2, 3 or 4, wherein
   R²¹ and R²³ are independently selected from hydrogen and (C₁-C₃)-alkyl and R²² is selected from the group consisting of (C₁-C₃)-alkoxy, (C₁-C₃)-alkyl and hydrogen, or
   R²² and R²³ together form a 5 atom heterocycle, optionally substituted by a group NHCO—(C₁-C₄)-alkyl;
or R¹⁴, R¹⁵ and carbons (α) and (β) bearing R¹⁴ and R¹⁵ together form

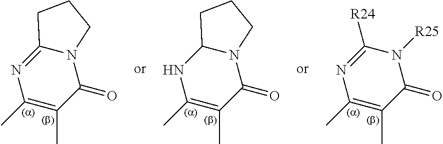

wherein R²⁴ and R²⁵ are independently selected from the group consisting of (C₁-C₃)-alkyl and hydrogen, preferably from hydrogen and methyl;
R¹⁶ is selected from hydrogen, (C₁-C₃)-alkoxy and halogen;
R¹⁷ is in ortho-position to R₁₆ and is selected from hydrogen, (C₁-C₃)-alkoxy and hydroxyl or form together with R¹⁷ a —O—CH₂—O-unit;
R¹⁸ and R¹⁹ are independently selected from the group consisting of hydrogen and halogen, preferably from H, F, Br and Cl, more preferably H or F;
   or any pharmaceutically acceptable salt thereof.

In a preferred embodiment, the new rocaglaol derivative of the formula (II) has one or several of the following features:
a) R¹¹ is alkoxy and R¹² is hydrogen;
b) R¹³ is selected from hydrogen and methoxy;
c) R¹⁴ is —NH—R²⁷, wherein R²⁷ is selected from —SO₂Me, —COR²⁸ with R²⁸ being H, methyl or ethyl;
d) R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂;
e) R¹⁶ is selected from hydrogen, (C₁-C₃)-alkoxy and halogen, more preferably from hydrogen, chlorine, bromine, fluorine and methoxy, still more preferably from bromine and methoxy;
f) R¹⁷ is hydrogen;
g) R¹⁸ is in para and is H or fluorine and R¹⁹ is hydrogen.

In particular, the rocaglaol derivative may present the formula (IIa) or (IIb). More preferably, the rocaglaol derivative has the formula (IIb) and R¹⁴ is —NH—R²⁷, wherein R²⁷ is —SO₂Me or —COR²⁸ with R²⁸ being H or (C₁-C₃)-alkyl, preferably H or methyl.

In a preferred embodiment, R¹¹ is alkoxy, especially methoxy or ethoxy, more preferably methoxy; R¹² is hydrogen; R¹³ is selected from hydrogen, methoxy and ethoxy, more preferably R¹³ is hydrogen or methoxy, still more preferably R¹³ is methoxy; R¹⁵ is selected from hydrogen, COOMe, CONH₂, CONHMe and CONMe₂, more preferably is hydrogen; R¹⁶ is selected from the group consisting of hydrogen, chlorine, bromine, fluorine and methoxy, more preferably is chlorine, bromine, or fluorine, still more preferably is bromine; and, R¹⁷, R¹⁸ and R¹⁹ are hydrogen.

In a particular embodiment, the rocaglaol derivative is selected from the group consisting of FL22, FL23, FL24 and FL25, preferably is FL23 or FL25.

In addition, the present invention provides a new rocaglaol derivative the formula (IIa), wherein

R¹² is H and R¹¹ is selected from methoxy and ethoxy, more preferably R¹¹ is methoxy;
R¹³ is selected from hydrogen, methoxy and ethoxy, more preferably R¹³ is hydrogen or methoxy;
R¹⁴ is —OCOR²⁰R²⁰ being selected from hydrogen, methyl and ethyl;
R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂; still more preferably R¹⁵ is hydrogen;
R¹⁶ is chlorine, bromine, or fluorine, preferably bromine;
R¹⁷ is hydrogen;
R¹⁸ is in para and is H or fluorine and R¹⁹ is hydrogen;
or any pharmaceutically acceptable salt thereof.

More preferably, R¹¹ and R¹³ are methoxy, R¹², R¹⁵, R¹⁸ and R¹⁹ are hydrogen, and R¹⁶ is bromine.

In a particular embodiment, the rocaglaol derivative is selected from the group consisting of FL19, FL20 and FL21.

Furthermore, the present invention provides a new rocaglaol derivative the formula (IIa), wherein

R¹² is H and R¹¹ is selected from methoxy and ethoxy, more preferably R¹¹ is methoxy;
R¹³ is selected from hydrogen, methoxy and ethoxy, more preferably R¹³ is hydrogen or methoxy;
R¹⁴ is hydroxyl;
R¹⁸ is in para and is fluorine and R¹⁹ is hydrogen,
and wherein
R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂;
R¹⁶ is bromine and R¹⁷ is hydrogen;
   or
R¹⁵ is selected from —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂; and,
R¹⁶ is methoxy and R¹⁷ is hydrogen;
   or any pharmaceutically acceptable salt thereof.

More preferably, R¹¹ and R¹³ are methoxy, R¹² is hydrogen, R¹⁶ is bromine and R¹⁵ is selected from hydrogen, COOMe, CONH₂, CONHMe and CONMe₂. In an alternative preferred embodiment, R¹¹ and R¹³ are methoxy, R¹² is hydrogen, R¹⁶ is methoxy and R¹⁵ is selected from COOMe, CONH₂, CONHMe and CONMe₂. Optionally, R¹¹ and R¹³ are methoxy, R¹² is hydrogen, and R¹⁵ is selected from COOMe, CONH₂, CONHMe and CONMe₂.

In a particular embodiment, the rocaglaol derivative is FL16, FL17 or FL18.

In a second aspect, the present invention concerns a new rocaglaol derivative according to the invention as a medicament.

In another aspect, the present invention concerns a pharmaceutical composition comprising a new rocaglaol derivative according to the present invention and a pharmaceutically acceptable carrier and/or excipient.

The present invention also concerns a pharmaceutical composition comprising a new rocaglaol derivative according to the present invention and an antineoplastic agent, preferably an anthracycline.

The present invention further concerns a product containing a new rocaglaol derivative according to the present invention and an antineoplastic agent, preferably an anthracycline, as a combined preparation for simultaneous, separate or sequential use in the treatment of cancer.

### LEGENDS OF THE FIGURES

FIG. 1 shows a schematic representation of the synthesis of racemic bromo-demethoxy-rocaglaol (FL3).

FIG. 2 shows a schematic representation of the synthesis of racemic rocaglaol (FL1) and its analogues (FL2 to FL4).

FIG. 3 shows a schematic representation of the synthesis of racemic flavaglines FL5 to FL11.

FIG. 4 shows a schematic representation of the synthesis of racemic flavaglines FL5 to FL10, and FL12 to FL18. Reactants and conditions: (a) hv, (E)-PhCH═CHCOOMe, CH₂Cl₂-MeOH, 0° C., 15 h; (b) MeONa, MeOH, 60° C., 20 min; (c) LiCl, H₂O, DMSO, 100° C., 12 h; (d) Me₄NBH(OAc)₃, AcOH, CH₃CN; (e) (i) KOH, MeOH, 45° C., 12 h, (ii) Me₂NH.HCl or MeNH₂.HCl, ECDI, HOBT, DIPEA, CH₂Cl₂, rt, 12 h; f) NH₃, MeOH, 100° C., 36 h; (g) hn, CH₂Cl₂-MeOH, 0° C., 15 h; (h) hv, CH₂Cl₂-MeOH, 0° C., 15 h.

FIG. 5 shows a schematic representation of the synthesis of racemic flavaglines FL19 to FL23. Reactants and conditions: (i) DCC, DMAP, HCOOH, CH₂Cl₂, rt, 36 h; (j) (R³CO)₂O, DMAP, pyr, rt, 6 h; (k) H₂NOH.HCl, pyr, EtOH, 70° C., 4 h; (l) LiAlH₄, THF, 45° C., 3 h; (m) HCOOEt, THF, reflux, 12 h.

FIG. 6 shows a schematic representation of the synthesis of racemic flavaglines FL22 to FL25. Reactants and conditions: (k) H₂NOMe.HCl, pyr, EtOH, 70° C., 4 h; (l) LiAlH₄, THF, 45° C., 3 h; (m) HCOOEt, THF, reflux, 12 h or MeSO₂Cl, N-methylmorpholine, CH₂Cl₂, rt.

FIG. 7 is a graph that shows the inhibition of cell proliferation of HepG2 cells exposed to FL1 and FL3 with or without 150 nM doxorubicin. HepG2 cells were treated with FL1 ( ) or FL3 (⋄) alone or with doxorubicin (FL1:▪, FL3:♦) for 72 h hours, and cell proliferation was measured by MTS assay. Experiments were performed in duplicate (n=3).

FIGS. 8A and 8B show the cardioprotective effect of FL1 (FIG. 8A) and FL3 (FIG. 8B) on apoptosis induced by doxorubicin (1 μM) in H9c2 cardioblast cells. These two graphs represent the relative total apoptotic cells obtained with doxorubicin alone or in combination with FL1 (FIG. 8A) or FL3 (FIG. 8B), in comparison with the results obtained with the vehicle.

FIG. 9 shows the cardioprotective effect of FL5, FL6, FL7 and FL8 on apoptosis induced by doxorubicin (1 μM) in H9c2 cells at different concentrations.

FIG. 10A shows a Western blot analysis demonstrating that doxorubicin induces caspase-3 activity (line 2) that is blocked by FL3 (1 nM) (line 3). Note that FL3 (line 4) and vehicle (line 1) do not induce caspase-3 activity. FIG. 10B is a histogram of caspase-3 activity according to Western blot quantification and normalization with GAPDH.

FIG. 11 shows the protection by FL3 of H9c2 cardiomyocytes against serum starvation. H9c2 were cultured in 1% serum in the presence of vehicle or FL3 (20 nM). Apoptosis was measured by FACS assay.

FIG. 12: FIG. 12A) FL3 prevents doxorubicin and KRIBB3 induced apoptosis. H9c2 cardiomyocytes were treated with doxorubicin (1 μM), or the HSP27 inhibitor KRIBB3 (1 μM) in presence or absence of FL3 (100 nM) for 1 h and then labeled with annexin V and PI for FACS analysis. FIG. 12B) Western blott analyses revealed that that FL3 maximally induces HSP27 phosphorylation (upper bands) without altering total HSP27 levels (lower band) in 1 h in H9c2 cells.

FIG. 13 shows the cardioprotective effect of FL1-10 and FL12-18 at 10 (left) or 100 (right) nM on apoptosis induced by doxorubicin (1 mM) in H9c2 cells.

### DETAILED DESCRIPTION OF THE INVENTION

The inventors have surprisingly found that flavaglines, in particular rocaglaol and some of its derivatives, may act as cardioprotectant agents. These compounds are found to be able to prevent or limit the apoptosis induced by the cardiotoxicity of antineoplastic agents such as anthracyclines, while potentiating the anti-tumor efficacy of these molecules.

Flavaglines are a family of natural compounds extracted from Asian plants of the genus Aglaia (Kim et al., 2006 and Proksch et al., 2001), comprising, for instance, rocaglaol, rocaglamide or silvestrol. Some of these molecules are known to inhibit the proliferation of tumor cells in a low nanomolar range, either by cytostatic (Hausott et al., 2004) or cytotoxic effects (Kim et al., 2007 and Mi et al., 2006), without displaying any significant toxicity on normal cells. Up to now, flavaglines have only been known to exhibit anticancer, immunosuppressive, neuroprotective (Fahrig et al., 2005), antifungal insecticide and anti-inflammatory (EP 1 693 059) properties.

The present invention provides new cardioprotectant rocaglaol derivatives of the formula (I)

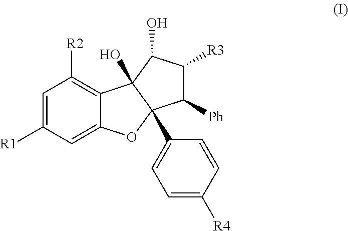

wherein
R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholin;
R² is selected from the group consisting of hydrogen and (C₁-C₃)-alkoxy, preferably from hydrogen and methoxy;
R³ is selected from the group consisting of —COOR⁵ and —CONH₂, wherein R⁵ is (C₁-C₃)-alkyl, preferably methyl; and
R⁴ is halogen, preferably bromine, chloride, iodide, more preferably bromine,
or
   wherein,
   R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholin;
   R² is hydrogen;
   R³ is selected from the group consisting of —COOR⁵ and —CONH₂, wherein R⁵ is (C₁-C₃)-alkyl, preferably methyl; and
   R⁴ is selected from the group consisting of hydrogen, halogen and alkoxy, preferably hydrogen or alkoxy,
or
   wherein,
   R¹ is substituted alkoxy, preferably a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine.
   R² is selected from the group consisting of hydrogen and (C₁-C₃)-alkoxy, preferably from hydrogen and methoxy; more preferably R² is C₁-C₃)-alkoxy, preferably methoxy;
   R³ is selected from the group consisting of —COOR⁵ and —CONH₂, wherein R⁵ is (C₁-C₃)-alkyl, preferably methyl, and
   R⁴ is selected from the group consisting of hydrogen, halogen and alkoxy, more preferably preferably hydrogen or alkoxy,
   or any pharmaceutically acceptable salt thereof.

In one embodiment, R¹ is selected from the group consisting of methoxy or O—(CH₂)ₙ—OH wherein n is 1, 2 or 3, preferably R¹ is a methoxy; R² is hydrogen; R³ is selected from the group consisting of —COOR⁵ and —CONH₂, wherein R⁵ is methyl; and, R⁴ is halogen or methoxy, preferably bromine or methoxy. In particular, the rocaglaol derivative may be FL5, FL7, and FL9.

In another embodiment, R¹ is a substituted alkoxy —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine, preferably R¹⁰ is hydroxyl; R² is hydrogen or methoxy, preferably hydrogen; R³ is selected from the group consisting of —COOR⁵ and —CONH₂, wherein R⁵ is (C₁-C₃)-alkyl, preferably methyl; and, R⁴ is halogen or methoxy, preferably bromine or methoxy.

In a particular embodiment, R¹ is a substituted alkoxy —O—(CH₂)ₙ—R¹⁰ wherein n is 2 and R¹⁰ is hydroxyl; R² is hydrogen; R³ is —COOR⁵, wherein R⁵ is methyl; and R⁴ is methoxy.

In another particular embodiment, R¹ is a substituted alkoxy —O—(CH₂)ₙ—R¹⁰ wherein n is 3 and R¹⁰ is hydroxyl; R² is hydrogen; R³ is —COOR⁵, wherein R⁵ is methyl; and R⁴ is methoxy.

In a particular embodiment, R³ is —CONH₂. In this embodiment, 1) R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholin; R² is selected from the group consisting of hydrogen and (C₁-C₃)-alkoxy, preferably from hydrogen and methoxy; and, R⁴ is halogen, preferably bromine, chloride, iodide, more preferably bromine; or 2) R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholin; R² is hydrogen; and R⁴ is selected from the group consisting of hydrogen, halogen and alkoxy, preferably hydrogen or alkoxy; or, 3) R¹ is substituted alkoxy, preferably a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine; R² is selected from the group consisting of hydrogen and (C₁-C₃)-alkoxy, preferably from hydrogen and methoxy; more preferably R² is C₁-C₃)-alkoxy, preferably methoxy; and, R⁴ is selected from the group consisting of hydrogen, halogen and alkoxy, more preferably hydrogen or alkoxy. In particular, the rocaglaol derivative may be FL9 and FL 14.

In another particular embodiment, R³ is —COOR⁵ with R⁵ is (C₁-C₃)-alkyl, preferably methyl. In this embodiment, R¹ is alkoxy, optionally substituted, preferably selected from the group consisting of methoxy and a group —O—(CH₂)ₙ—R¹⁰ wherein n is 1, 2, 3 or 4 and R¹⁰ is selected from the group consisting of hydroxyl, —NMe₂, —OCONMe₂, —OCONH₂ and morpholine; R² is hydrogen; and, R⁴ is selected from the group consisting of hydrogen, methoxy and halogen. Preferably, R³ is COOMe, R¹ is methoxy, R² is hydrogen and R⁴ is selected from the group consisting of hydrogen, methoxy and bromine, preferably is methoxy or bromine. In particular, the rocaglaol derivative may be FL5 or FL7.

In a more preferred embodiment, the present invention concerns a compound of formula (I) wherein R¹ is methoxy; R² is selected from the group consisting of methoxy or hydrogen; R³ is selected from the group consisting of —COOR⁵, and —CONH₂, wherein R⁵ is methyl; and, R⁴ is halogen, preferably bromine.

In a particular embodiment, R¹ is methoxy, R² is hydrogen, R³ is —CONH₂ and R⁴ is bromine (FL9).

In another preferred embodiment, R¹ is methoxy; R² is hydrogen; R³ is —COOR⁵, wherein R⁵ is methyl; and, R⁴ is halogen or methoxy, preferably bromine or methoxy. In a particular embodiment, R¹ is methoxy; R² is hydrogen; R³ is —COOR⁵, wherein R⁵ is methyl; and R⁴ is bromine (FL5).

In another particular embodiment, R¹ is methoxy; R² is hydrogen; R³ is —COOR⁵, wherein R⁵ is methyl; and R⁴ is methoxy (FL7).

In another aspect, the present invention also concerns a new rocagloal derivative of the formula (I), wherein

R¹ is methoxy;
R² is selected from the group consisting of hydrogen and (C₁-C₃)-alkoxy, preferably from hydrogen and methoxy;
R³ is selected from the group consisting of hydrogen, —COOR⁵ and —CONR⁶R⁷, wherein
R⁵ is (C₁-C₃)-alkyl, preferably methyl, and
R⁶ and R⁷ are independently selected from the group consisting of hydrogen and (C₁-C₃)-alkyl, preferably from hydrogen and methyl; and
R⁴ is bromine,
or any pharmaceutically acceptable salt thereof.

Preferably, it concerns a rocagloal derivative of the formula (I), wherein

R¹ is methoxy; and R⁴ is bromine,
and wherein
R² is (C₁-C₃)-alkoxy, preferably methoxy, and,
R³ is hydrogen,
   or
R² is hydrogen, and,
R³ is selected from the group consisting of hydrogen, —COOR⁵ and —CONR⁶R⁷, wherein R⁵ is (C₁-C₃)-alkyl, preferably methyl, and R⁶ and R⁷ are independently selected from the group consisting of hydrogen and (C₁-C₃)-alkyl, preferably from hydrogen and methyl;
   or any pharmaceutically acceptable salt thereof.

In a preferred embodiment, R¹ and R² are methoxy, R³ is hydrogen and R⁴ is bromine (FL3). In a further preferred embodiment, R¹ and R² are methoxy, R³ is —CONH₂, and R⁴ is bromine (FL14).

In another embodiment, R¹ is methoxy; R² is hydrogen; R³ is selected from the group consisting of hydrogen, —COO(CH₃) and —CON(CH₃)₂, preferably of hydrogen and —CON(CH₃)₂; and R⁴ is bromine. In a preferred embodiment, R¹ is methoxy; R² is hydrogen; R³ is —CON(CH₃)₂ and R⁴ is bromine (FL8). In another preferred embodiment, R¹ is methoxy; R² is hydrogen; R³ is hydrogen and R⁴ is bromine (FL6).

In a further aspect, the present invention also concerns a new rocagloal derivative of the formula (II), wherein
R¹¹ is alkoxy, optionally substituted;
R¹² is hydrogen;
or R¹¹ and R¹² together form a —O—CH₂—O-unit;
R¹³ is selected from hydrogen and (C₁-C₃)-alkoxy;
R¹⁴ is —NH—R²⁷, wherein R²⁷ is selected from —SO₂Me, —COR²⁸ with R²⁸ being H or (C₁-C₃)-alkyl;
R¹⁵ is selected from hydrogen, —COOR²¹, —CONR²²R²³ and —CONH(CH₂)ₙOH, wherein n is 2, 3 or 4, wherein
   R²¹ and R²³ are independently selected from hydrogen and (C₁-C₃)-alkyl and R²² is selected from the group consisting of (C₁-C₃)-alkoxy, (C₁-C₃)-alkyl and hydrogen, or
   R²² and R²³ together form a 5 atom heterocycle, optionally substituted by a group NHCO—(C₁-C₄)-alkyl;
or R¹⁴, R¹⁵ and carbons (α) and (β) bearing R¹⁴ and R¹⁵ together form

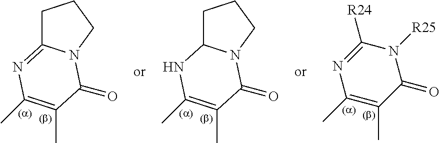

wherein R²⁴ and R²⁵ are independently selected from the group consisting of (C₁-C₃)-alkyl and hydrogen, preferably from hydrogen and methyl;
R¹⁶ is selected from hydrogen, (C₁-C₃)-alkoxy and halogen;
R¹⁷ is in ortho-position to R₁₆ and is selected from hydrogen, (C₁-C₃)-alkoxy and hydroxyl or form together with R¹⁷ a —O—CH₂—O-unit;
R¹⁸ and R¹⁹ are independently selected from the group consisting of hydrogen and halogen, preferably from H, F, Br and Cl, more preferably H or F;
   or any pharmaceutically acceptable salt thereof.

In this embodiment, the rocaglaol derivative of the formula (II) has preferably one or several of the following features:
a) R¹¹ is alkoxy and R¹² is hydrogen;
b) R¹³ is selected from hydrogen and methoxy;
c) R¹⁴ is —NH—R²⁷, wherein R²⁷ is selected from —SO₂Me, —COR²⁸ with R²⁸ being H, methyl or ethyl;
d) R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂;
e) R¹⁶ is selected from hydrogen, (C₁-C₃)-alkoxy and halogen, more preferably from hydrogen, chlorine, bromine, fluorine and methoxy, still more preferably from bromine and methoxy;
f) R¹⁷ is hydrogen;
g) R¹⁸ is in para and is H or fluorine and R¹⁹ is hydrogen.

Optionally, the rocaglaol derivative of the formula (II) meets one feature, two features [for instance a) and b); a) and c); a) and d); a) and e); a) and f); a) and g); b) and c); b) and d); b) and e); b) and f); b) and g); c) and d); c) and e); c) and f); c) and g); d) and e); d) and f); d) and g); e) and f); e) and g); f) and g)], three features [for instance a), b) and c); a), b) and d); a), b) and e); a), b) and f); a), b) and g); a), c) and d); a), c) and e); a), c) and f); a), c) and g); a), d) and e); a), d) and f); a), d) and g); a), e) and f); a), e) and g); b), c) and d); b), c) and e); b), c) and f); b), c) and g); c), d) and e); c), d) and f); c), d) and g); d), e) and f); d), e) and g); e), f) and g)], four features [a), b), c) and d); a), b), c) and e); a), b), c) and f); a), b), c) and g); a), b), d) and e); a), b), d) and f); a), b), d) and g); a), b), e) and f); a), b), e) and g); a), b), f) and g); a), c), d) and e); a), c), d) and f); a), c), d) and g); a), c), e) and f), a), c), e) and g); a), d), e) and f); a), d), e) and g); a), d), f) and g); a), e), f) and g); b), c), d) and e); b), c), d) and f); b), c), d) and g); b), d), e) and f); b), d), e) and g); b), e), f) and g); c), d), e) and f); c), d), e) and g); c), e), f) and g); d), e), f) and g)], five features [for instance a), b), c), d) and e); a), b), c), d) and f); a), b), c), d) and g); a), b), c), e) and f); a), b), c), e) and g); a), b), c), f) and g); a), b), d), e) and f); a), b), d), e) and g); a), b), d), f) and g); a), b), e), f) and g); a), c), d), e) and f); a), c), d), e) and g); a), c), d), f) and g); a), c), e), f) and g); a), d), e), f) and g); b), c), d), e) and f); b), c), d), e) and g); b), c), e), f) and g); b), d), e), f) and g); c), d), e), f) and g)], six features [a), b), c), d), e) and f); a), b), c), d), e) and g); a), b), c), d), f) and g); a), b), c), e), f) and g); a), b), d), e), f) and g); a), c), d), e), f) and g); b), c), d), e), f) and g)] or all the seven features a), b), c), d), e), f) and g).

In particular, the rocaglaol derivative may present the formula (IIa) or (IIb), the definition of R11, R12, R13, R14, R15, R16, R17, R18 and R19 is the same as above.

In a preferred embodiment, the rocaglaol derivative has the formula (IIb) and R¹⁴ is —NH—R²⁷, wherein R²⁷ is —SO₂Me or —COR²⁸ with R²⁸ being H or (C₁-C₃)-alkyl, preferably H or methyl. More preferably, R¹¹ is alkoxy, especially methoxy or ethoxy, more preferably methoxy; R¹² is hydrogen; R¹³ is selected from hydrogen, methoxy and ethoxy, more preferably R¹³ is hydrogen or methoxy, still more preferably R¹³ is methoxy; R¹⁵ is selected from hydrogen, COOMe, CONH₂, CONHMe and CONMe₂, more preferably is hydrogen; R¹⁶ is selected from the group consisting of hydrogen, chlorine, bromine, fluorine and methoxy, more preferably is chlorine, bromine, or fluorine, still more preferably is bromine; and, R¹⁷, R¹⁸ and R¹⁹ are hydrogen. In a preferred embodiment, the rocaglaol of formula (IIb) is such as R¹¹ and R¹³ are methoxy, R¹⁵ is hydrogen, R¹⁶ is bromine, and, R¹⁷, R¹⁸ and R¹⁹ are hydrogen. In this particular preferred embodiment, R¹⁴ can be —NHCHO (FL23) or —NHSO₂Me (FL25). Alternatively, the rocaglaol of formula (IIa) is such as R¹¹ and R¹³ are methoxy, R¹⁵ is hydrogen, R¹⁶ is bromine, R¹⁷, R¹⁸ and R¹⁹ are hydrogen, and R¹⁴ can be —NHCHO (FL22) or —NHSO₂Me (FL24).

In a further aspect, the present invention also concerns a new rocagloal derivative of the formula (II), wherein

R¹² is H and R¹¹ is selected from methoxy and ethoxy, more preferably R¹¹ is methoxy;
R¹³ is selected from hydrogen, methoxy and ethoxy, more preferably R¹³ is hydrogen or methoxy;
R¹⁴ is —OCOR²⁰, R²⁰ being selected from hydrogen, methyl and ethyl;
R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂; still more preferably R¹⁵ is hydrogen;
R¹⁶ is chlorine, bromine, or fluorine, preferably bromine;
R¹⁷ is hydrogen;
R¹⁸ is in para and is H or fluorine and R¹⁹ is hydrogen;
or any pharmaceutically acceptable salt thereof.

More preferably, R¹¹ and R¹³ are methoxy, R¹², R¹⁵, R¹⁸ and R¹⁹ are hydrogen, and R16 is bromine. Still preferably, R¹⁴ is —OCHO (FL19), —OCOMe (FL20) or —OCOEt (FL21).

The present invention finally provides a new rocaglaol derivative the formula (IIa), wherein

R¹² is H and R¹¹ is selected from methoxy and ethoxy, more preferably R¹¹ is methoxy;
R¹³ is selected from hydrogen, methoxy and ethoxy, more preferably R¹³ is hydrogen or methoxy;
R¹⁴ is hydroxyl;
R¹⁸ is in para and is fluorine and R¹⁹ is hydrogen,
and wherein
R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂;
R¹⁶ is bromine and R¹⁷ is hydrogen;
   or
R¹⁵ is selected from —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂; and,
R¹⁶ is methoxy and R¹⁷ is hydrogen;
   or any pharmaceutically acceptable salt thereof.

More preferably, in this embodiment, R¹¹ and R¹³ are methoxy, R¹² is hydrogen, R¹⁶ is bromine and R¹⁵ is selected from hydrogen, COOMe, CONH₂, CONHMe and CONMe₂. Still more preferably, R¹⁵ is COOMe (FL17) or CONH₂ (FL18). In an alternative preferred embodiment, R¹¹ and R¹³ are methoxy, R¹² is hydrogen, R¹⁶ is methoxy and R¹⁵ is selected from COOMe, CONH₂, CONHMe and CONMe₂. Still more preferably, R¹⁵ is COOMe (FL 16).

Optionally, R¹¹ and R¹³ are methoxy, R¹² is hydrogen, and R¹⁵ is selected from COOMe, CONH₂, CONHMe and CONMe₂.

As used herein, a “cardioprotectant agent” is an agent that protects cardiomyocytes from damages, in particular from apoptosis.

The term “alkoxy”, as used herein, corresponds to an alkyl group bonded to the molecule by an —O— (ether) bond. (C₁-C₃)-alkoxy groups include methoxy, ethoxy, propyloxy, and isopropyloxy.

The term “alkyl” as used herein, refers to a univalent radical containing only carbon and hydrogen atoms arranged in a chain. (C₁-C₃)-alkyl groups include methyl, ethyl, propyl, or isopropyl. The term “Me” as used herein, refers to a methyl group. The term “Et” as used herein, refers to an ethyl group.

The term “morpholin” as used herein, refers to a heterocycle of formula —O(CH₂CH₂)₂NH with both amine and ether functional groups.

The term “heterocycle” as used herein, refers to a group containing a ring structure comprising one or more heteroatoms, such as sulfur, oxygen or nitrogen. 5 atom heterocycles include, for example, furan, pyrrole and oxazole.

The term “pharmaceutically acceptable salt” refers to salts which are non-toxic for a patient and suitable for maintaining the stability of a therapeutic agent and allowing the delivery of said agent to target cells or tissue. Pharmaceutically acceptable salts are well known in the art (Berge et al., 1977). These salts can be prepared in situ during the final isolation and purification of the rocaglaol derivatives, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, 3-phenylpropionate, phosphate, picrate, pivalate, propionate, stearate, succinate, sulfate, tartrate, thiocyanate, p-toluenesulfonate, undecanoate, valerate salts, and the like.

The present invention also concerns a rocaglaol derivative of the formulae (I), (II), (IIa) and (IIb) according to the invention, as described above, as a medicament, in particular as cardioprotectant agent, and more particularly as cardioprotectant agent against cardiotoxicity of an antineoplastic agent. In particular, the derivative is selected in the group consisting of FL3, FL5, FL6, FL7, FL8, FL9, FL14, FL16, FL17, FL18, FL19, FL20, FL21, FL22, FL23, FL24 and FL25.

Those derivatives have also demonstrated advantageous cytotoxicity against tumoral cells. Accordingly, the present invention further concerns a rocaglaol derivative of the formulae (I), (II), (IIa) and (IIb) according to the invention for use for treating cancer. It also concerns the use of a rocaglaol derivative of the formulae (I), (II), (IIa) and (IIb) according to the invention for preparing a medicament for treating cancer. It finally concerns a method for treating a cancer in a subject in need thereof, comprising administering a therapeutically active amount of a rocaglaol derivative of the formulae (I), (II), (IIa) and (IIb) according to the invention. This particular aspect also concerns the preferred embodiments disclosed above for the rocaglaol derivatives of the formulae (I), (II), (IIa) and (IIb), and in particular a derivative selected in the group consisting of FL3, FL5, FL6, FL7, FL8, FL9, FL14, FL16, FL17, FL18, FL19, FL20, FL21, FL22, FL23, FL24 and FL25.

These rocaglaol derivatives can be synthesized by any process known by the man skilled in the art. Such processes are described, for example, in the patent applications DE 19934952 and WO 2005/092876 or in the articles of Diedrichs et al. (Diedrichs et al., 2005), Gerard et al. (Gerard et al., 2004) and Dobler et al. (Dobler et al., 2001). As illustrative example, the synthesis of twenty three of these rocaglaol derivatives, including bromo-demethoxy-rocaglaol (FL3) and methyl bromo-didemethoxy-rocaglate (FL5), is detailed in the present application in examples 1, 2 and 3.

The present invention also concerns a pharmaceutical composition comprising a cardioprotectant rocaglaol derivative of formulae (I), (II), (IIa) and (IIb) according to the present invention and a pharmaceutically acceptable carrier and/or excipient. This particular aspect also concerns the preferred embodiments disclosed above for the rocaglaol derivatives of the formulae (I), (II), (IIa) and (IIb), and in particular a derivative selected in the group consisting of FL3, FL5, FL6, FL7, FL8, FL9, FL14, FL16, FL17, FL18, FL19, FL20, FL21, FL22, FL23, FL24 and FL25.

The pharmaceutical composition comprising the rocaglaol derivative of formulae (I), (II), (IIa) and (IIb) according to the present invention, including their preferred embodiments and the compounds FL3, FL5, FL6, FL7, FL8, FL9, FL14, FL16, FL17, FL18, FL19, FL20, FL21, FL22, FL23, FL24 and FL25, is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Possible pharmaceutical compositions include those suitable for oral, rectal, topical (including transdermal, buccal and sublingual), or parenteral (including subcutaneous, intramuscular, intravenous and intradermal) administration. For these formulations, conventional excipient can be used according to techniques well known by those skilled in the art.

The compositions for parenteral administration are generally physiologically compatible sterile solutions or suspensions which can optionally be prepared immediately before use from solid or lyophilized form. Adjuvants such as a local anesthetic, preservative and buffering agents can be dissolved in the vehicle and a surfactant or wetting agent can be included in the composition to facilitate uniform distribution of the active ingredient.

For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

In a preferred embodiment, the pharmaceutical composition of the invention is suitable for parenteral administration.

Pharmaceutical composition according to the invention may be formulated to release the active drug substantially immediately upon administration or at any predetermined time or time period after administration.

Pharmaceutical composition according to the invention can comprise one or more cardioprotectant rocaglaol derivatives associated with pharmaceutically acceptable excipients and/or carriers. These excipients and/or carriers are chosen according to the form of administration as described above. Other active compounds can also be associated with rocaglaol derivatives of the present invention such as other cardioprotectant molecules or antineoplastic agents, such as anthracycline, preferably doxorubicin.

The present invention also concerns a product containing a rocaglaol derivative of formulae (I), (II), (IIa) and (IIb) according to the present invention, including their preferred embodiments and the compounds FL3, FL5, FL6, FL7, FL8, FL9, FL14, FL16, FL17, FL18, FL19, FL20, FL21, FL22, FL23, FL24 and FL25, and an antineoplastic agent, preferably an anthracycline, as a combined preparation for simultaneous, separate or sequential use in the treatment of cancer.

The present invention also concerns the use of a rocaglaol derivative of the formula (II)

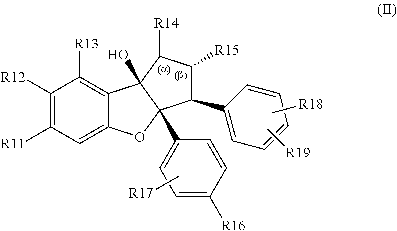

wherein
R¹¹ is alkoxy, optionally substituted;
R¹² is hydrogen;
or R¹¹ and R¹² together form a —O—CH₂—O-unit;
R¹³ is selected from hydrogen and (C₁-C₃)-alkoxy;
R¹⁴ is selected from hydroxyl, oxo group, —OCOR²⁰, R²⁰ being selected from hydrogen and (C₁-C₃)-alkyl, ═N—OR²⁶ with R²⁶ being H or methyl, —NH—(CH₂)ₘ—R²⁷, wherein m is 0 or 1 and R²⁷ is selected from H, —OH, —SO₂Me, —COR²⁸ with R²⁸ being H or (C₁-C₃)-alkyl;
R¹⁵ is selected from hydrogen, —COOR²¹, —CONR²²R²³ and —CONH(CH₂)ₙOH, wherein n is 2, 3 or 4, wherein
R²¹ and R²³ are independently selected from hydrogen and (C₁-C₃)-alkyl and R²² is selected from the group consisting of (C₁-C₃)-alkoxy, (C₁-C₃)-alkyl and hydrogen, or
R²² and R²³ together form a 5 atom heterocycle, optionally substituted by a group NHCO—(C₁-C₄)-alkyl;
or R¹⁴, R¹⁵ and carbons (α) and (β) bearing R¹⁴ and R¹⁵ together form

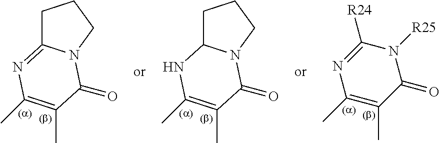

wherein R²⁴ and R²⁵ are independently selected from the group consisting of (C₁-C₃)-alkyl and hydrogen, preferably from hydrogen and methyl;
R¹⁶ is selected from hydrogen, (C₁-C₃)-alkoxy and halogen;
R¹⁷ is in ortho-position to R₁₆ and is selected from hydrogen, (C₁-C₃)-alkoxy and hydroxyl or form together with R¹⁷ a —O—CH₂—O-unit;
R¹⁸ and R¹⁹ are independently selected from the group consisting of hydrogen and halogen, preferably from H, F, Br and Cl, more preferably H or F;
   or any pharmaceutically acceptable salt thereof, for use for preventing or limiting the cardiotoxicity of an antineoplastic agent, preferably an anthracycline, more preferably doxorubicin.

Preferably, the rocaglaol derivative of the formula (II) has one or several of the following features:
a) R¹² is H and R¹¹ is selected from methoxy and ethoxy, more preferably R¹¹ is methoxy;
b) R¹³ is selected from hydrogen, methoxy and ethoxy, more preferably R¹³ is hydrogen or methoxy;
c) R¹⁴ is selected from hydroxyl, —OCOR²⁰, R²⁰ being selected from hydrogen, methyl and ethyl, —NH—(CH₂)ₘ—R²⁷, wherein m is 0 and R²⁷ is —SO₂Me or —COR²⁸ with R²⁸ being H or (C₁-C₃)-alkyl, preferably H or methyl;
d) R¹⁵ is selected from hydrogen, —COOR²¹, and —CONR²²R²³, wherein R²¹ and R²³ are independently selected from hydrogen and methyl, and R²² is selected from the group consisting of hydrogen, methyl and methoxy; more preferably R¹⁵ is selected from hydrogen, COOMe, CONH₂, CONHMe and CONMe₂;
e) R¹⁶ is selected from hydrogen, (C₁-C₃)-alkoxy and halogen, more preferably from hydrogen, chlorine, bromine, fluorine and methoxy, still more preferably from bromine and methoxy;
f) R¹⁷ is hydrogen;
g) R¹⁸ is in para and is H or fluorine and R¹⁹ is hydrogen.

Optionally, the rocaglaol derivative of the formula (II) meets one feature, two features [for instance a) and b); a) and c); a) and d); a) and e); a) and f); a) and g); b) and c); b) and d); b) and e); b) and f); b) and g); c) and d); c) and e); c) and f); c) and g); d) and e); d) and f); d) and g); e) and f); e) and g); f) and g)], three features [for instance a), b) and c); a), b) and d); a), b) and e); a), b) and f); a), b) and g); a), c) and d); a), c) and e); a), c) and f); a), c) and g); a), d) and e); a), d) and f); a), d) and g); a), e) and f); a), e) and g); b), c) and d); b), c) and e); b), c) and f); b), c) and g); c), d) and e); c), d) and f); c), d) and g); d), e) and f); d), e) and g); e), f) and g)], four features [a), b), c) and d); a), b), c) and e); a), b), c) and f); a), b), c) and g); a), b), d) and e); a), b), d) and f); a), b), d) and g); a), b), e) and f); a), b), e) and g); a), b), f) and g); a), c), d) and e); a), c), d) and f); a), c), d) and g); a), c), e) and f), a), c), e) and g); a), d), e) and f); a), d), e) and g); a), d), f) and g); a), e), f) and g); b), c), d) and e); b), c), d) and f); b), c), d) and g); b), d), e) and f); b), d), e) and g); b), e), f) and g); c), d), e) and f); c), d), e) and g); c), e), f) and g); d), e), f) and g)], five features [for instance a), b), c), d) and e); a), b), c), d) and f); a), b), c), d) and g); a), b), c), e) and f); a), b), c), e) and g); a), b), c), f) and g); a), b), d), e) and f); a), b), d), e) and g); a), b), d), f) and g); a), b), e), f) and g); a), c), d), e) and f); a), c), d), e) and g); a), c), d), f) and g); a), c), e), f) and g); a), d), e), f) and g); b), c), d), e) and f); b), c), d), e) and g); b), c), e), f) and g); b), d), e), f) and g); c), d), e), f) and g)], six features [a), b), c), d), e) and f); a), b), c), d), e) and g); a), b), c), d), f) and g); a), b), c), e), f) and g); a), b), d), e), f) and g); a), c), d), e), f) and g); b), c), d), e), f) and g)] or all the seven features a), b), c), d), e), f) and g).

Optionally, R¹⁸ is a halogen and is in position para, ortho or meta and R¹⁹ is hydrogen. Preferably, R¹⁸ is F, Cl or Br, more preferably F or Br, still more preferably is F. Preferably, R¹⁸ is in para. Optionally, R¹⁸ and R¹⁹ are both halogens, preferably the same halogen, and R¹⁸ and R¹⁹ are in para and ortho, in ortho and meta, in para and meta, or both in ortho. Preferably, R¹⁸ and R¹⁹ are both F, Cl or Br, more preferably F.

In a particular embodiment, the rocaglaol derivative presents the formula (IIa) or (IIb)

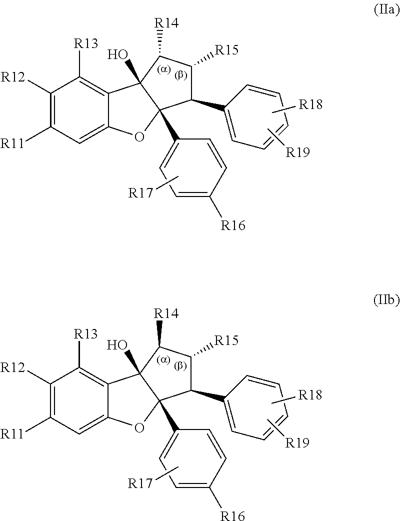

wherein the definition of R11, R12, R13, R14, R15, R16, R17, R18 and R19 is the same than for formula (II) and the same preferred features also apply to the compounds of formula (IIa) or (IIb).

In a particular preferred embodiment, the rocaglaol derivative has the formula (IIa) and R¹⁴ is selected from hydroxyl, —OCOR²⁰, R²⁰ being selected from hydrogen and (C₁-C₃)-alkyl, and ═N—OR²⁶ with R²⁶ being H or methyl. Preferably, R¹⁴ is selected from hydroxyl and —OCOR²⁰, R²⁰ being selected from hydrogen, methyl and ethyl.

In a preferred embodiment of this particular embodiment,
R¹¹ is alkoxy, especially methoxy or ethoxy, more preferably methoxy;
R¹² is hydrogen;
R¹³ is selected from hydrogen, methoxy and ethoxy, more preferably R¹³ is hydrogen or methoxy;
R¹⁵ is selected from hydrogen, COOMe, CONH₂, CONHMe and CONMe₂;
R¹⁶ is selected from the group consisting of hydrogen, chlorine, bromine, fluorine and methoxy, more preferably of bromine and methoxy;
R¹⁸ is in para and is H or fluorine; and,
R¹⁷ and R¹⁹ are hydrogen.

Optionally, the rocaglaol derivative has the formula (III)

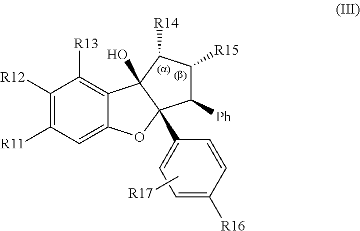

wherein
R¹¹ is alkoxy, optionally substituted; and R¹² is hydrogen;
or R¹¹ and R¹² together form a —O—CH₂—O-unit;
R¹³ is selected from the group consisting of hydrogen and (C₁-C₃)-alkoxy, preferably from hydrogen and methoxy;
R¹⁴ is selected from the group consisting of hydroxyl, oxo group and —OCOR²⁰, R²⁰ being selected from (C₁-C₃)-alkyl and hydrogen, preferably from hydrogen and methyl;
R¹⁵ is selected from the group consisting of hydrogen, —COOR²¹, —CONR²²R²³ and —CONH(CH₂)ₙOH, wherein n is 2, 3 or 4, wherein
R²¹ and R²³ are independently selected from hydrogen and (C₁-C₃)-alkyl, preferably from hydrogen and methyl, and R²² is selected from the group consisting of (C₁-C₃)-alkoxy, (C₁-C₃)-alkyl and hydrogen, preferably from hydrogen, methyl and methoxy, or
R²² and R²³ together form a 5 atom heterocycle, optionally substituted by a group NHCO—(C₁-C₄)-alkyl;
or R¹⁴, R¹⁵ and carbons (α) and (β) bearing R¹⁴ and R¹⁵ together form

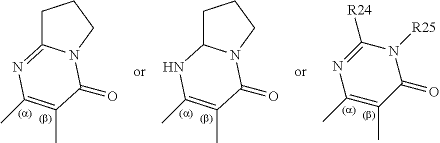

wherein R²⁴ and R²⁵ are independently selected from the group consisting of (C₁-C₃)-alkyl and hydrogen, preferably from hydrogen and methyl;
R¹⁶ is selected from the group consisting of hydrogen, (C₁-C₃)-alkoxy and halogen;
R¹⁷ is in ortho-position to R₁₆ and is selected from the group consisting of hydrogen, (C₁-C₃)-alkoxy and hydroxyl or form together with R¹⁷ a —O—CH₂—O-unit;
or any pharmaceutically acceptable salt thereof.

In another particular preferred embodiment, the rocaglaol derivative has the formula (IIb) and R¹⁴ is —NH—(CH₂)ₘ—R²⁷, wherein m is 0 and R²⁷ is —SO₂Me or —COR²⁸ with R²⁸ being H or (C₁-C₃)-alkyl, preferably H or methyl.

In a preferred embodiment of this particular embodiment,
R¹¹ is alkoxy, especially methoxy or ethoxy, more preferably methoxy;
R¹² is hydrogen;
R¹³ is selected from hydrogen, methoxy and ethoxy, more preferably R¹³ is hydrogen or methoxy, still more preferably R¹³ is methoxy;
R¹⁵ is selected from hydrogen, COOMe, CONH₂, CONHMe and CONMe₂, more preferably is hydrogen;
R¹⁶ is selected from the group consisting of hydrogen, chlorine, bromine, fluorine and methoxy, more preferably is chlorine, bromine, or fluorine, still more preferably is bromine; and,
R¹⁷, R¹⁸ and R¹⁹ are hydrogen.

Examples of suitable rocaglaol derivatives of the formula (II) are disclosed in the following documents: WO07/139,749; EP1693059; WO05/113529; WO05/092876; WO03/045375; U.S. Pat. No. 6,518,274; U.S. Pat. No. 6,420,393; DE19934952.

The present invention also concerns a rocaglaol derivative of the formulae (I), (II), (IIa), (IIb) and (III) as described above or any pharmaceutically acceptable salt thereof, for preventing or limiting the cardiotoxicity of an antineoplastic agent. Preferably, this particular aspect also concerns the preferred embodiments disclosed above for the rocaglaol derivatives of the formulae (I), (II), (IIa) and (IIb), and in particular a derivative selected in the group consisting of FL1, FL3, FL5, FL6, FL7, FL8, FL9, FL10, FL12, FL13, FL14, FL15, FL16, FL17, FL18, FL19, FL20, FL21, FL22, FL23, FL24 and FL25, more preferably from the group consisting of FL1, FL3, FL5, FL8, FL9, FL10, FL12, FL13, FL14, FL15, FL16, FL17, FL18, FL19, FL20, FL21, FL22, FL23 and FL25.

The present invention further concerns a method for preventing or limiting the cardiotoxicity of an antineoplastic agent in a subject comprising administering a therapeutically effective amount of a rocaglaol derivative of the formulae (I), (II), (IIa), (IIb) and (III) as described above or any pharmaceutically acceptable salt thereof to said subject. This particular aspect also concerns the preferred embodiments disclosed above for the rocaglaol derivatives of the formulae (I), (II), (IIa) and (IIb), and in particular a derivative selected in the group consisting of FL1, FL3, FL5, FL6, FL7, FL8, FL9, FL10, FL12, FL13, FL14, FL15, FL16, FL17, FL18, FL19, FL20, FL21, FL22, FL23, FL24 and FL25, more preferably from the group consisting of FL1, FL3, FL5, FL8, FL9, FL10, FL12, FL13, FL14, FL15, FL16, FL17, FL18, FL19, FL20, FL21, FL22, FL23 and FL25. The subject is any mammal, preferably a human being. In a particular embodiment, the method of the invention for preventing or limiting the cardiotoxicity of an antineoplastic agent comprises administering 0.01 to 10 mg/kg of body weight/day of a rocaglaol derivative of the formulae (I), (II), (IIa), (IIb) and (III) as described above or any pharmaceutically acceptable salt thereof to said subject. Preferably, the method of the invention comprises administering 0.1 to 5 mg/kg of body weight/day of a rocaglaol derivative of the formulae (I), (II), (IIa), (IIb) and (III) as described above or any pharmaceutically acceptable salt thereof to said subject. This particular aspect also concerns the preferred embodiments disclosed above for the rocaglaol derivatives of the formulae (I), (II), (IIa) and (IIb), and in particular a derivative selected in the group consisting of FL1, FL3, FL5, FL6, FL7, FL8, FL9, FL10, FL12, FL13, FL14, FL15, FL16, FL17, FL18, FL19, FL20, FL21, FL22, FL23, FL24 and FL25, more preferably from the group consisting of FL1, FL3, FL5, FL8, FL9, FL10, FL12, FL13, FL14, FL15, FL16, FL17, FL18, FL19, FL20, FL21, FL22, FL23 and FL25.

The term “cardiotoxicity”, “cardiovascular toxicity” or “cardiotoxic side effect” means having a toxic effect on the heart, which includes cardiac events, such as but not limited to, mild blood pressure changes, thrombosis, electrocardiographic (ECG) changes, arrthymias, myocarditis, pericarditis, myocardial infarction (MI), cardiomyopathy, cardiac failure (left ventricular dysfunction or failure) and congestive heart failure (CHF). Considering the cardiotoxicity of antineoplastic agents, these side effects are essentially due to apoptosis of cardiomyocytes induced by such agents.

An “antineoplastic agent” is an agent with anti-cancer activity that inhibits or halts the growth of cancerous cells or immature pre-cancerous cells, kills cancerous cells or immature pre-cancerous cells, increases the susceptibility of cancerous or pre-cancerous cells to other antineoplastic agents, and/or inhibits metastasis of cancerous cells. These agents may include chemical agents as well as biological agents. Various antineoplastic agents can cause cardiovascular toxicity such as anthracyclines, cyclophosphamide, paclitaxel, fluorouracil, rituximab, arsenic trioxide, trastuzumab, thalidomide, etoposide, vinca alkaloids, pentastatin, cytarabine, interferons, busulfan and cisplatin.

Preferably, the antineoplastic agent belongs to the group of anthracyclines. In the present document, “anthracyclines” include but are not limited to, doxorubicin, daunorubicin, idarubicin, detorubicin, caminomycin, epirubicin, morpholinodoxorubicin, morpholinodaunorubicin, methoxymorpholinyldoxorubicin, substitutes, derivatives, and one or more combinations thereof. More preferably, the anthracycline molecule is doxorubicin. As used herein, the term “to prevent or to limit the cardiotoxicity” means inhibiting or reducing cardiotoxic side effects of the antineoplastic agent administered to the subject, in particular, inhibiting or reducing the apoptosis of cardiomyocytes. The effect of the rocaglaol derivative on the apoptosis of cardiomyocytes can be assessed by any method known by the skilled person. For example, in the experimental section, the effects of compounds according to invention are assessed by measuring and comparing the apoptosis of H9c2 cardiomyocytes in absence and in presence of an antineoplastic agent and with a combination of an antineoplastic agent and a rocaglaol derivative of the invention. Results are expressed in percentage of apoptotic cells. Preferably, the apoptosis of cardiomyocyte in presence of a rocaglaol derivative of the invention is reduced by at least 5%, more preferably by at least 10% and the most preferably by at least 25%.

The present invention also concerns the use of a rocaglaol derivative of the formulae (I), (II), (IIa), (IIb) and (III) as described above, or any of its pharmaceutically acceptable salts, as cardioprotectant agent. This particular aspect also concerns the preferred embodiments disclosed above for the rocaglaol derivatives of the formulae (I), (II), (IIa) and (IIb), and in particular a derivative selected in the group consisting of FL1, FL3, FL5, FL6, FL7, FL8, FL9, FL10, FL12, FL13, FL14, FL15, FL16, FL17, FL18, FL19, FL20, FL21, FL22, FL23, FL24 and FL25, more preferably from the group consisting of FL1, FL3, FL5, FL8, FL9, FL10, FL12, FL13, FL14, FL15, FL16, FL17, FL18, FL19, FL20, FL21, FL22, FL23 and FL25.

Preferably, the rocaglaol derivative is one of the formulae (I), (II), (IIa), (IIb) according to the invention, as described herein.

In a particular embodiment, the rocaglaol derivative is selected from the group consisting of FL1, FL3, FL5, FL6, FL7, FL8, FL9, FL10, FL12, FL13, FL14, FL15, FL16, FL17, FL18, FL19, FL20, FL21, FL22, FL23, FL24 and FL25, more preferably from the group consisting of FL1, FL3, FL5, FL8, FL9, FL10, FL12, FL13, FL14, FL15, FL16, FL17, FL18, FL19, FL20, FL21, FL22, FL23 and FL25.

The amount of cardioprotectant rocaglaol derivative of the invention to be administered has to be determined by standard procedure well known by those of ordinary skill in the art. Physiological data of the patient (e.g. age, size, and weight) and the routes of administration have to be taken into account to determine the appropriate dosage. The cardioprotectant rocaglaol derivative of the invention may be administered as a single dose or in multiple doses.

In a preferred embodiment, the pharmaceutical composition comprises a therapeutically effective amount of the rocaglaol derivative of the invention.

By a “therapeutically effective amount” is intended an amount of cardioprotectant rocaglaol derivative of the invention administered to a patient that is sufficient to provide a cardioprotective effect, i.e. that is sufficient to protect cardiomyocytes from damages, in particular from apoptosis.

This therapeutically effective amount comprises a blood plasma concentration in a range of from 1 nM to 1 μM, preferably of from 15 nM to 100 nM or from 18 nM to 90 nM, more preferably of from 20 nM to 70 nM, most preferably of from 23 nM to 50 nM, and even most preferably of from 25 nM to 30 nM. Then, it is to be understood that the total amount of the rocaglaol derivative of the invention may vary depending on the volume of blood plasma of the patient. Suitable means and measures to determine the therapeutically effective amount are available to the person skilled in the art.

In a particular embodiment, the pharmaceutical composition according to the invention comprises 0.1 mg to 1 g of the rocaglaol derivative of the invention. In another particular embodiment, the pharmaceutical composition according to the invention comprises 0.7 to 700 mg of the rocaglaol derivative of the invention, preferably 0.7 to 350 mg of the rocaglaol derivative of the invention.

Cardioprotectant rocaglaol derivatives of the invention can be used in combination with other cardioprotective molecules or with antineoplastic agents. In this case, rocaglaol derivatives and the other active molecules can be administered simultaneously or consecutively. Accordingly, the present invention discloses a pharmaceutical composition comprising a rocaglaol derivative of formula (II), preferably of formula (I), and an antineoplastic agent. It also concerns a product containing a rocaglaol derivative of formula (II), preferably of formula (I) and an antineoplastic agent as a combined preparation for simultaneous, separate or sequential use in the treatment of cancer. In a preferred embodiment, the antineoplastic agent is an anthracycline, in particular doxorubicin.

The following examples are given for purposes of illustration and not by way of limitation.

### EXAMPLES

### Example 1

### Synthesis of bromo-demethoxy-rocaglaol (FL3)

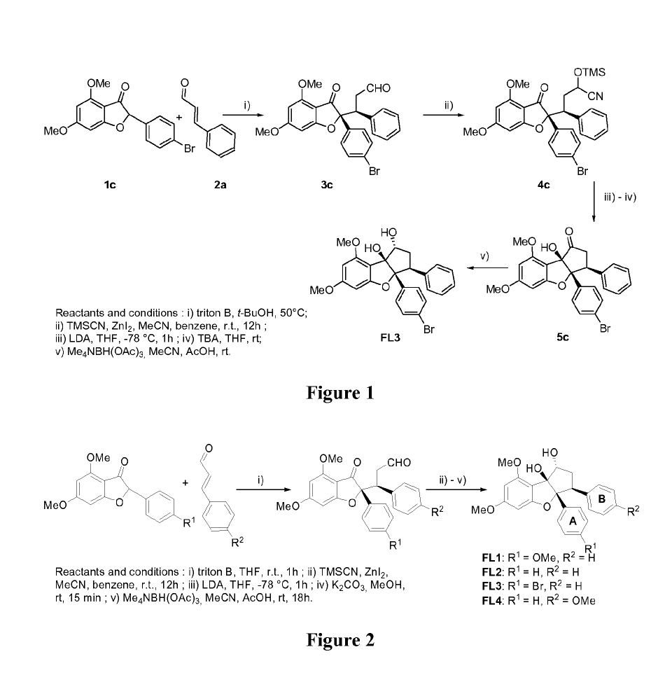

The synthesis of bromo-demethoxy-rocaglaol (FL3) is represented on FIG. 1. The different steps are described below.

### (S)-3-((R)-2,3-dihydro-4,6-dimethoxy-2-(4-bromophenyl)-3-oxobenzofuran-2-yl)-3-phenylpropanal (3c)

A suspension of benzofuranone 1c (4.7 g, 13.5 mmol) in t-BuOH (300 ml) was heated to 50° C. under argon. Benzyltrimethylammonium hydroxide in MeOH (40%, 306 μL, 0.73 mmol) and, immediately after, cinnamaldehyde 2a (3.40 ml, 27.0 mmol), were added. The mixture was stirred for 2 h at 50° C., cooled to room temperature (rt), concentred and acidified with HCl 1 M (30 ml), extracted with CH₂Cl₂, dried over MgSO₄ and concentrated to dryness. Purification of the resulting yellow solid residue by chromatography (Et₂O-Pentane 6:4) yielded 1.94 g (33%) of ketoaldehyde 3c as a white solid: R_{f} 0.4 (Et₂O/Hept 9:1). NMR ¹H (300 MHz, CDCl₃): 2.61 (1H, ddd, J=0.9, 4.0, 17.3 Hz), 3.07 (1H, ddd, J=2.30, 10.9, 17.3 Hz), 3.70 (3H, s), 3.85 (3H, s), 4.19 (1H, dd, J=4.0, 10.9 Hz), 5.81 (1H, d, J=1.8 Hz), 6.21 (1H, d, J=1.8 Hz), 7.08-7.17 (3H, m), 7.29-7.32 (2H, m), 7.49, 7.63 (4H, AA′BB′, J=8.7 Hz), 9.41 (1H, d, J=1.1 Hz). NMR ¹³C (75 MHz, CDCl₃): 44.0; 46.8; 55.9; 55.9; 88.4; 93.0; 103.7; 122.6; 126.9; 127.5; 128.2; 129.5; 131.8; 132.0; 135.6; 136.3; 159.1; 169.9; 174.1; 194.1; 200.0. IR (thin film): 2725, 1720, 1699, 1617, 1590, 1154, 747 cm⁻¹. HR-MS calcd for C₂₅H₂₁BrLiO₅: 487.0732. found: 487.0727 (−0.02 ppm).

### (S)-3-((R)-2,3-dihydro-4,6-dimethoxy-2-(4-bromophenyl)-3-oxobenzofuran-2-yl)-3-phenyl-2-[(trimethylsily)oxy]-propanenitrile (4c)

Acetonitrile (12 mL) was added at room temperature under argon to aldehyde 3c (1.2 g, 2.5 mmol). Trimethylsilyl cyanide (744 mg, 7.5 mmol) was added dropwise and immediately after zinc iodide (10 mg) was added at room temperature under argon. The resulting mixture was stirred for 1 hour, filtered and concentred. Cyanohydrin 4c (1.74 g) was used in the next step without purification. R_{f} 0.5 (Et₂O/Hept 8:2). NMR ¹H (300 MHz, CDCl₃): 7.47-7.60 (4H, m); 7.13-1.23 (5H, m); 6.21 (1H, t, J=2.3 Hz); 5.79 (1H, t, J=2.3 Hz); 3.87-3.89 (1H, m); 3.83 (3H, s); 3.70-3.76 (1H, m); 3.66 (3H, s); 2.30-2.38 (2H, m); −0.01 (9H, d, J=4.5 Hz).

### Tricyclic Ketone (5c)

LDA (2.7 mmol, 0.6 M) was added dropwise at −78° C. under argon to a solution of protected cyanohydrin 4c (1.43 g, 2.46 mmol) in dry THF (12 mL). After stirring for 2 hours at −78° C. the solution was heated to −50° C. for 10 min. The reaction was quenched by the addition of saturated aqueous ammonium chloride (15 mL). Standard extractive work-up (CH₂Cl₂) gave a yellow solid (1.49 g). This solid was directly treated with tetra-n-butylammonium fluoride (2.7 mL, 1M in THF) added dropwise at room temperature in dry THF (10 mL). The solution was stirred for 4 hours and she was quenched by addition of methanol. Standard extractive work-up (AcOEt) and purification by flash chromatography (Et₂O) afforded tricyclic ketone 5c (240 mg, 20%) as a white solid: R_{f} 0.35 (Et₂O/Hept 8:2). NMR ¹H (300 MHz, CDCl₃): 2.96-3.09 (2H, m), 3.21 (1H, br s), 3.81 (3H, s), 3.84 (3H, s), 3.90 (1H, dd, J=10.2, 12.1 Hz), 6.1 (1H, d, J=1.9 Hz), 6.33 (1H, d, J=1.9 Hz), 6.89-6.94 (4H, m), 7.10-7.12 (3H, m), 7.24-7.27 (2H, m). NMR ¹³C (75 MHz, CDCl₃): 39.6; 48.6; 55.6; 55.7; 88.6; 89.7; 92.8; 100.8; 106.2; 121.7; 127.1; 127.8; 128.0; 128.3; 130.7; 133.0; 136.6; 158.4; 160.9; 164.8; 210.3. IR (thin film): 3477, 2942, 2841, 1750, 1621, 1597, 1149 cm⁻¹. HR-MS calcd for C₂₅H₂₁Br₁Na₁O₆: 503.0470. found: 503.0465 (−0.99 ppm).

### Racemic bromo-demethoxy-rocaglaol (FL3)

Acetonitrile (1.8 mL) was added at room temperature under argon to tetramethylammonium triacetoxyborohydride (828 mg, 3.15 mmol) followed by the addition of glacial acetic acid (1.8 mL). After stirring for 30 min at room temperature a solution of ketone 5c (170 mg, 0.354 mmol) in dry acetonitrile (4.5 mL) was added dropwise. The resulting mixture was stirred overnight at room temperature. The reaction was quenched by the addition of saturated aqueous ammonium chloride (40 mL). Standard extractive work-up (AcOEt) and purification by flash chromatography (Et₂O) afforded FL3 (103 mg, 60%) as a white solid: R_{f}0.25 (Et₂O/Hept 8:2).

### Example 2

### Synthesis of methyl bromo-didemethoxy-rocaglate (FL5)

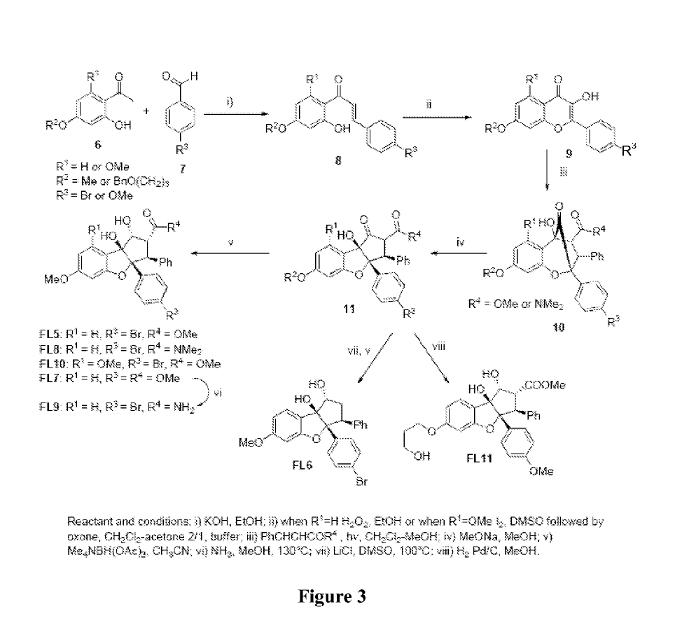

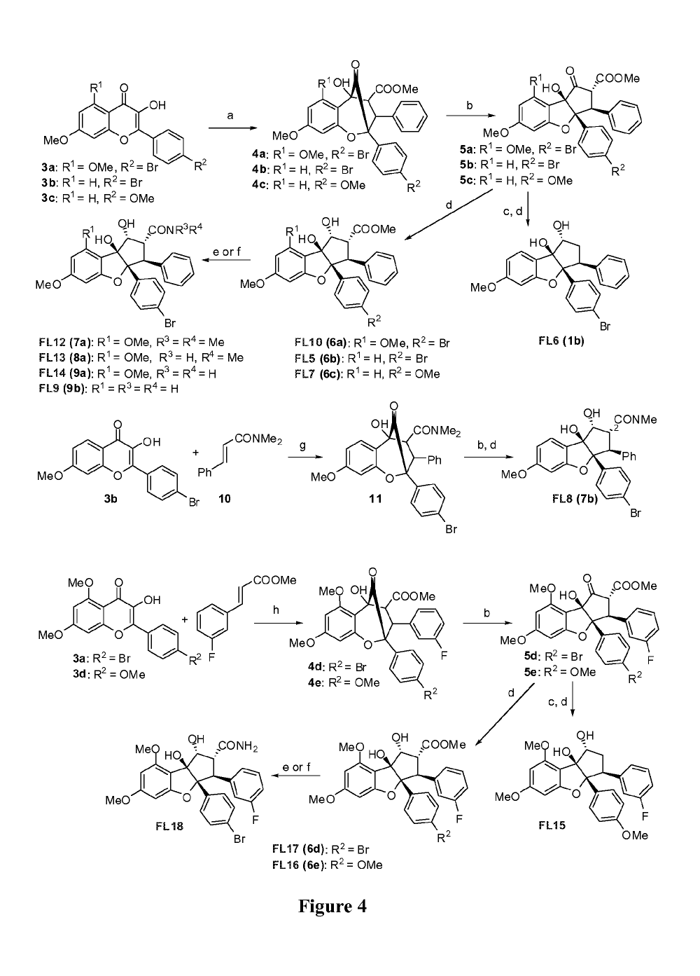

The synthesis of methyl bromo-didemethoxy-rocaglate (FL5) represented on FIGS. 3 and 4 is based on Porco's Strategy (Gerard et al., 2004). The different steps are described below.

### (E)-3-(4-bromophenyl)-1-(2-hydroxy-4-methoxyphenyl)prop-2-en-1-one (FIG. 3 (8): R¹═H, R²=Me, R³=Br)

To a solution of 10.2 g (0.062 mol) of 2′-hydroxy-4′-methoxyacetophenone in 150 mL of methanol, were successively added 13.8 g (4 eq, 0.25 mol) of potassium hydroxide and 11.4 g (1 eq, 0.062 mol) of 4-bromobenzaldehyde. The solution was heated overnight at 60° C., cooled to 0° C. in an ice bath and acidified to pH 2 with concentrated HCl. The precipitate was filtered, washed with 100 mL of water and dried in vacuo to afford 20.4 g (99%) of chalcone (8 of FIG. 3). NMR ¹H (300 MHz, CDCl₃): 3.83 (3H, s,), 6.45 (2H, m), 7.50 (5H, m), 7.77 (2H, m), 13.33 (1H, s). NMR ¹³C (75 MHz, CDCl₃): 55.8, 101.3, 108.1, 114.2, 121.1, 125.1, 130.0, 131.4, 132.4, 133.9, 143.1, 166.6, 167.0, 191.7.

### 2-(4-bromophenyl)-3-hydroxy-7-methoxy-4H-chromen-4-one (FIG. 3 (9): R¹═H, R²=Me, R³═Br)

To a solution of 10.0 g (1 eq, 0.030 mol) of chalcone (8 of FIG. 3) in 100 mL of methanol, were added 10.1 g (6 eq, 0.18 mol) of potassium hydroxide. The solution was heated at 60° C., and when it became red, 12.3 mL (4 eq, 0.12 mol) of hydrogene peroxide were slowly added. The solution was stirred for 15 min at r.t and cooled to 0° C. in an ice bath. The mixture was then acidified to pH 2 with concentrated HCl. The precipitate was filtered, washed with 100 mL of water and dried in vacuo to afford 7.30 g (70%) of 3-hydroxyflavone (9 of FIG. 3) as a yellow solid. NMR ¹H (300 MHz, CDCl₃): 3.92 (3H, s₂), 6.92 (1H, d, J=2.2 Hz), 6.98 (1H, dd, J=2.2; 8.8 Hz), 7.63 (2H, d, J=8.8 Hz), 8.11 (3H, m). NMR ¹³C (75 MHz, CDCl₃): 56.0, 100.2, 114.7, 115.1, 122.9, 126.0, 129.1, 130.6, 131.4, 139.1, 143.2, 156.4, 163.7, 172.3.

### Cyclopenta[bc]benzopyran (FIG. 3 (10): R¹═H, R²=Me, R³═Br) or Methyl 2-(4-bromophenyl)-5-hydroxy-2,5-methano-8-methoxy-10-oxo-3-phenyl-2,3,4,5-tetrahydro-1-benzoxepin-4-carboxylate (4b of FIG. 4)

A solution of 3-hydroxyflavone (9 of FIG. 3 or 3b of FIG. 4) (1 g, 1 eq, 3 mmol) and methyl cinnamate (5.15 g, 10 eq, 30 mmol) in 90 mL of a mixture of dichloromethane/methanol (3/1) in a pyrex tube was degassed with argon for 5 min, and irradiated for 15 h at 0° C. using an Iwasaki 400 W mercury lamp. The solution was concentrated in vacuo and purified by flash chromatography (40/60 heptane/AcOEt) to afford 560 mg of cyclopenta[bc]benzopyrans as a white solid, which was heated at 65° C. for 4 h in 20 ml, of AcOEt. The solution was concentrated in vacuo to afford 560 mg (38%) of cyclopenta[bc]benzopyran 10 as a white solid. NMR ¹H (300 MHz, CDCl₃): 3.80 (3H, s), 3.82 (3H, m), 3.70 (1H, d, J=8.3 Hz), 4.65 (1H, d, J=8.3 Hz), 6.56 (2H, m), 7.19-7.40 (9H, m), 7.52 (1H, d, J=8.4 Hz). NMR ¹³C (75 MHz, CDCl₃): 52.8, 54.4, 55.8, 60.9, 88.6, 97.8, 97.3, 101.9, 108.4, 117.0, 122.5, 125.7, 127.4, 128.5, 130.0, 130.8, 134.7, 139.3, 152.6, 161.7, 171.5, 191.5.

### Cyclopenta[b]tetrahydrobenzofuran (FIG. 3 (11): R¹═H, R²=Me, R³═Br) or Methyl 3a-(4-bromophenyl)-8b-hydroxy-8-methoxy-1-oxo-3-phenyl-2,3-3a,8b-tetrahydro-cyclopenta[b]benzofuran-2-carboxylate (5b of FIG. 4)

To a solution of cyclopenta[bc]benzopyran (10 of FIG. 3 or 4b of FIG. 4) (400 mg, 1 eq, 0.8 mmol) in MeOH (75 mL) were added 78 mg (2,5 eq, 2.0 mmol) of NaH in MeOH (5 mL) at 0° C. The resulting solution was stirred for 20 min at 60° C. After quenching the reaction with saturated aqueous NH₄Cl, 40 mL of AcOEt was then added, and the organic layer was washed with water (2×20 mL) and brine (20 mL). The organic layer was dried over MgSO₄ and concentrated in vacuo to afford 370 mg of crude ketol (93%) as a white solide which was used without further purification. NMR ¹H (300 MHz, CDCl₃): 3.67 (3H, s), 3.87 (3H, s), 4.11 (1H, s), 4.54 (1H, s, H₆), 6.64 (1H, dd, J=2.3, 8.4 Hz), 6.72 (1H, d, J=2.3 Hz), 6.90-7.40 (10H, m). NMR ¹³C(CDCl₃): 51.8, 53.2, 55.8, 55.9, 87.7, 96.9, 100.6, 108.4, 115.3, 118.0, 126.0, 127.8, 128.3, 128.4, 128.7, 129.0, 132.8, 138.5, 145.0, 159.9, 172.2, 203.9.

### Methyl bromo-didemethoxy-rocaglate or Methyl 3a-(4-bromophenyl)-1,8b-dihydroxy-8-methoxy-1-oxo-3-phenyl-2,3-3a,8b-tetrahydro-cyclopenta[b]benzofuran-2-carboxylate (6b of FIG. 4) (FL5)

To a solution of 620 mg (6 eq, 2.35 mmol) of Me₄NBH(OAc)₃ and 0.23 mL (10 eq, 3.93 mmol) of acetic acid in 20 mL of CH₃CN was added a solution of 200 mg (1 eq, 0.39 mmol) of the crude ketol product (5b of FIG. 4) in 10 mL of CH₃CN. The resulting solution was stirred for 3 h at rt before being quenched with 20 mL of saturated aqueous NH₄Cl. The solution was then treated with 10 mL of a 3 M aqueous solution of sodium/potassium tartrate and stirred for 30 min. The aqueous solution was extracted with CH₂Cl₂ (2×30 mL). The combined organic layers were washed with brine, dried over MgSO₄ and concentrated in vacuo. HPLC purification afforded 100 mg (50%) of the corresponding endo product as a white solid.

### Example 3

### Synthesis and NMR Characterization of Rocaglaol Derivatives FL6 to FL25

### Synthesis of FL10

### Methyl 2-(4-bromophenyl)-6,8-dimethoxy-5-hydroxy-2,5-methano-10-oxo-3-phenyl-2,3,4,5-tetrahydro-1-benzoxepin-4-carboxylate (4a of FIG. 4)

A solution of hydroxyflavone (3a of FIG. 4) (1.0 g, 2.7 mmol) and methyl cinnamate (3.43 g, 21 mmol) in 90 mL of CH₂Cl₂/MeOH (3:1) was degassed with argon for 10 min in a pyrex tube. This mixture was then irradiated (450 W Iwasaki UV lamp) for 30 h at 0° C. under an argon atmosphere. The solution was concentrated in vacuo, purified by flash chromatography (heptane/AcOEt 8:2 to 4:6), heated to reflux in EtOAc (20 mL) for 4 h and concentrated in vacuo to give 400 mg (29%) of 4a of FIG. 4 as a white solid. NMR ¹H(CDCl₃): 3.56 (3H, s), 3.76 (3H, s), 3.83 (3H, s), 4.17 (1H, d, J=9.1 Hz), 4.49 (1H, d, J=9.1 Hz), 6.09 (1H, d, J=2.1 Hz), 6.19 (1H, d, J=2.1 Hz), 6.90-7.20 (5H, m), 7.21 (2H, d, J=9.1 Hz), 7.50 (2H, d, J=9.1 Hz). NMR ¹³C(CDCl₃): 51.9, 52.4, 53.5, 54.6, 55.4, 55.6, 56.0, 56.3, 57.2, 62.3, 81.0, 81.3, 83.8, 87.5, 92.9, 93.9, 94.6, 98.0, 103.7, 104.7, 121.9, 122.1, 126.9, 127.5, 128.1, 128.5, 128.9, 129.0, 129.3, 129.8, 130.4, 130.5, 130.7, 131.3, 132.6, 134.8, 137.5, 139.4, 152.7, 153.5, 158.2, 158.6, 161.5, 162.2, 170.6, 171.7, 202.9.

### Methyl 3a-(4-bromophenyl)-6,8-dimethoxy-8b-hydroxy-1-oxo-3-phenyl-2,3-3a,8b-tetrahydro-cyclopenta[b]benzofuran-2-carboxylate (5a of FIG. 4)

To a solution of 4a of FIG. 4 (120 mg, 0.2 mmol) in MeOH (15 mL) was added a solution of NaOMe in MeOH (0.1 M, 5 mL) at 0° C. The resulting solution was stirred for 20 min at 60° C., cooled to rt, and quenched with saturated NH4Cl, extracted with AcOEt (20 mL), and the organic layer was washed with water (2×20 mL) and brine (20 mL), dried over MgSO₄ and concentrated in vacuo to afford 120 mg of crude β-ketoester 5a of FIG. 4 as white solid which was used without further purification. NMR ¹H(CDCl₃): 3.65 (3H, s), 3.77 (3H, s), 3.84 (3H, s), 4.05 (1H, d, J=12.7 Hz), 4.24 (1H, d, J=12.7 Hz), 4.47 (1H, s), 6.10 (1H, d, J=2.0 Hz), 6.33 (1H, d, J=2.0 Hz), 6.90-7.20 (5H, m), 7.27 (2H, d, J=8.7 Hz), 7.50 (2H, d, J=8.7 Hz). NMR ¹³C(CDCl₃): 51.9, 53.1, 55.7, 55.8, 56.9, 88.5, 89.2, 92.7, 99.1, 105.9, 122.0, 127.0, 127.8, 128.2, 128.5, 130.9, 134.3, 135.1, 158.7, 160.7, 165.1, 170.0, 203.1.

### Methyl 3a-(4-bromophenyl)-1,8b-dihydroxy-6,8-dmethoxy-1-oxo-3-phenyl-2,3-3a,8b-tetrahydro-cyclopenta[b]benzofuran-2-Carboxylate (FL10 or 6a of FIG. 4)

Glacial acetic acid (128 mL, 2.22 mmol) was added to a solution of Me₄NBH(OAc)₃ (351 mg, 1.33 mmol) in CH₃CN (3 mL). After stirring for 5 min at room temperature, a solution of 5a of FIG. 4 (120 mg, 0.22 mmol) in CH₃CN (10 mL) was added dropwise. The resulting mixture was stirred was stirred for 3 h at rt, successively quenched with saturated aqueous NH₄Cl (15 mL) and a 3 M aqueous solution of sodium/potassium tartrate (3 mL) and stirred for 30 min. The aqueous solution was extracted with AcOEt (2×30 mL). The combined organic layers were washed with brine, dried over MgSO₄ and concentrated in vacuo. Purification by HPLC (Symetry shield RP18, 7 μm, 19×300 mm, with a flow rate of 10 mL/min using a 50 min gradient from water (0.1% TFA) to CH₃CN (0.1% TFA)) yielded diol 6a of FIG. 4 (60 mg, 50%) as a white solid. NMR data: see below.

### Synthesis of FL6 (1b of FIG. 4)

### 3a-(4-Bromophenyl)-8b-hydroxy-8-methoxy-3-phenyl-2,3-3a,8b-tetrahydro-cyclopenta[b]benzofuran-1-one

Lithium chloride (13 mg, 0.3 mmol) and water (11 μL, 0.6 mmol) were added to a solution of 5b of FIG. 4 (100 mg, 0.2 mmol) in DMSO. The mixture was stirred at 100° C. for 12 h, cooled to room temperature, diluted with 10 mL of water and extracted with AcOEt (3×10 mL). The combined organic layers were washed with brine, dried over MgSO₄ and concentrated. The white solid was purified by flash chromatography (heptane/AcOEt 9:1 to 4:6), and concentrated in vacuo to give 40 mg (22%) of the title ketone as a white solid. ¹H NMR (CDCl₃): 3.05 (2H, m), 3.80 (1H, m), 3.90 (3H, s), 6.67 (1H, dd, J=2.3, 8.5 Hz), 6.75 (1H, d, J=2.3 Hz), 6.87-6.94 (4H, m), 7.10-7.14 (3H, m), 7.24-7.27 (3H, m). ¹³C NMR (CDCl₃): 39.5, 52.8, 55.6, 88.7, 90.1, 92.6, 100.8, 106.2, 121.4, 126.5, 126.8, 127.8, 128.0, 128.8, 130.7, 133.8, 137.3, 160.9, 164.8, 210.5.

### 3a-(4-Bromophenyl)-8-methoxy-3-phenyl-1,2,3,3a-tetrahydro-cyclopenta[b]benzofuran-1,8b(1H)-diol (FL6, 1b of FIG. 4)

Glacial acetic acid (51 μL, 0.89 mmol) was added to a solution of Me₄NBH(OAc)₃ (140 mg, 0.53 mmol) in CH₃CN (3 mL). After stirring for 5 min at room temperature, a solution of the previous compound (40 mg, 0.09 mmol) in CH₃CN (2 mL) was added dropwise. The resulting mixture was stirred for 3 h at rt, successively quenched with saturated aqueous NH₄Cl (15 mL) and a 3 M aqueous solution of sodium/potassium tartrate (3 mL) and stirred for 30 min. The aqueous solution was extracted with AcOEt (2×30 mL). The combined organic layers were washed with brine, dried over MgSO₄ and concentrated in vacuo. Purification by HPLC (Symetry shield RP18, 7 μm, 19×300 mm, with a flow rate of 10 mL/min using a 50 min gradient from water (0.1% TFA) to CH₃CN (0.1% TFA)) yielded diol 1b of FIG. 4 (FL6) (20 mg, 50%) as a white solid. NMR data: see below.

### Synthesis of FL 7 (6c of FIG. 4)

### Methyl 5-hydroxy-2,5-methano-8-methoxy-2-(4-methoxyphenyl)-10-oxo-2,3,4,5-tetrahydro-1-benzoxepin-4-carboxylate (4c of FIG. 4)

A solution of hydroxyflavone 3c of FIG. 4 (1.0 g, 3.4 mmol) and methyl cinnamate (5.15 g, 34 mmol) in 90 mL of CH₂Cl₂/MeOH (3:1) was degassed with argon for 10 min in a pyrex tube. This mixture was then irradiated (450 W Iwasaki UV lamp) for 15 h at 0° C. under an argon atmosphere. The solution was concentrated in vacuo, purified by flash chromatography (heptane/AcOEt 4:6), heated to reflux in EtOAc (20 mL) for 4 h and concentrated in vacuo to give 440 mg (29%) of adduct 4c of FIG. 4 as a white solid. ¹H NMR (CDCl₃): 3.62 (1H, d, J=8.1 Hz), 3.68 (3H, s), 3.73 (3H, s), 3.83 (3H, s), 4.65 (1H, d, J=8.1 Hz), 6.60 (2H, m), 7.10-7.40 (8H, m), 7.56 (2H, d, J=8.9 Hz). ¹³C NMR (CDCl₃): 50.7, 52.8, 54.4, 55.8, 55.9, 88.4, 98.0, 101.7, 108.2, 117.3, 125.5, 127.3, 128.6, 130.2, 130.9, 134.6, 139.1, 152.8, 161.6, 162.6, 171.4, 191.4.

### Methyl 8b-hydroxy-8-methoxy-3a-(4-methoxyphenyl)-1-oxo-3-phenyl-2,3-3a,8b-tetrahydro-cyclopenta[b]benzofuran-2-carboxylate (5c of FIG. 4)

To a solution of aglain 4c of FIG. 4 (900 mg, 0.8 mmol) in MeOH (100 mL) was added a solution of NaOMe in MeOH (0.4 M, 5 mL) at 0° C. The resulting solution was stirred for 20 min at 60° C., cooled to rt, and quenched with saturated NH₄Cl, extracted with AcOEt (40 mL), and the organic layer was washed with water (2×20 mL) and brine (20 mL), dried over MgSO₄ and concentrated in vacuo to afford 370 mg of crude β-ketoester 5c of FIG. 4 as a white solid which was used without further purification. ¹H NMR (CDCl₃): 3.69 (3H, s), 3.77 (3H, s), 3.91 (3H, s), 4.16 (1H, s), 4.57 (1H, s), 6.75 (2H, m), 6.90-7.20 (8H, m), 7.56 (2H, d, J=8.4 Hz). ¹³C NMR (CDCl₃): 50.9, 51.9, 53.4, 55.6, 56.0, 87.6, 96.7, 100.9, 108.6, 115.5, 117.9, 127.8, 128.2, 128.3, 128.8, 129.1, 132.7, 138.3, 145.3, 160.1, 162.5, 172.2, 203.9.

### Methyl 1,8b-dihydroxy-8-methoxy-3a-(4-methoxyphenyl)-1-oxo-3-phenyl-2,3-3a,8b-tetrahydro-cyclopenta[b]benzofuran-2-carboxylate (FL7, 6c of FIG. 4)

Glacial acetic acid (248 mL, 4.33 mmol) was added to a solution of Me₄NBH(OAc)₃ (686 mg, 2.61 mmol) in CH₃CN (30 mL). After stirring for 5 min at room temperature, a solution of ketone 5c of FIG. 4 (200 mg, 0.43 mmol) in CH₃CN (10 mL) was added dropwise. The resulting mixture was stirred for 3 h at rt, successively quenched with saturated aqueous NH₄Cl (20 mL) and a 3 M aqueous solution of sodium/potassium tartrate (10 mL) and stirred for 30 min. The aqueous solution was extracted with CH₂Cl₂ (2×30 mL). The combined organic layers were washed with brine, dried over MgSO₄ and concentrated in vacuo. Purification by HPLC (Symetry shield RP18, 7 μm, 19×300 mm, with a flow rate of 10 mL/min using a 50 min gradient from water (0.1% TFA) to CH₃CN (0.1% TFA)) yielded diol 6c of FIG. 4 (100 mg, 50%) as a white solid. NMR data: see below.

### Synthesis of FL9, FL12, FL 13 and FL14 (7a, 8a, 9a and 9b of FIG. 4)

### 3a-(4-Bromophenyl)-1,8b-dihydroxy-6,8-dimethoxy-N,N-dimethyl-1-oxo-3-phenyl-2,3-3a,8b-tetrahydro-cyclopenta[b]benzofuran-2-carboxamide (FL12, 7a of FIG. 4)

To a solution of acid 6a of FIG. 4 (30 mg, 0.06 mmol) in CH₂Cl₂ (3 mL) was added dimethylamine hydrochloride (6 mg, 0.07 mmol) and DMAP (8 mg, 0.07 mmol). At 0° C., EDCI (13 mg, 0.07 mmol) was added portionwise over a 5 min period. The mixture was stirred for 30 min at 0° C. and then triethylamine (9 μL, 0.07 mmol) was added. The solution was stirred 1 h at 0° C. and 15 h at rt, quenched with 1 mL of 1N HCl and diluted with 5 mL of water. The aqueous layer was extracted with CH₂Cl₂ (3×15 mL). The collected organic layers were washed with brine, dried over MgSO₄, concentrated, purified by flash chromatography (CH₂Cl₂/AcOEt/Et₃N 78:20:2 to 58:40:2), and concentrated in vacuo to afford 20 mg (63%) of amide 7a of FIG. 4 (FL12) as a white solid. NMR data: see below.

### 3a-(4-Bromophenyl)-1,8b-dihydroxy-6,8-dimethoxy-N-methyl-1-oxo-3-phenyl-2,3-3a,8b-tetrahydro-cyclopenta[b]benzofuran-2-carboxamide (FL13, 8a of FIG. 4)

To a solution of acid 6a of FIG. 4 (30 mg, 0.06 mmol) in CH₂Cl₂ (3 mL) was added methylamine hydrochloride (5 mg, 0.07 mmol) and DMAP (8 mg, 0.07 mmol). At 0° C., EDCI (13 mg, 0.07 mmol) was added portionwise over a 5 min period. The mixture was stirred for 30 min at 0° C. and then triethylamine (9 μL, 0.07 mmol) was added. The solution was stirred 1 h at 0° C. and 15 h at rt. The mixture was quenched with 1 mL of 1N HCl and diluted with 5 mL of water. The aqueous layer was extracted with CH₂Cl₂ (3×15 mL). The collected organic layer was washed with brine (20 mL), dried over MgSO₄, concentrated, purified by flash chromatography (CH₂Cl₂/AcOEt/Et₃N 68:30:2 to 58:40:2), and concentrated in vacuo to afford 26 mg (84%) of amide 8a of FIG. 4 (FL13) as a white solid. NMR data: see below.

### 3a-(4-Bromophenyl)-1,8b-dihydroxy-6,8-dimethoxy-1-oxo-3-phenyl-2,3-3a,8b-tetrahydro-cyclopenta[b]benzofuran-2-carboxamide (FL14, 9a of FIG. 4)

Ester 6a of FIG. 4 (50 mg, 0.09 mmol) was added to a saturated solution of ammoniac in methanol (5 mL) in a sealed tube. The solution was then heated at 100° C. for 36 h, cooled to rt, and concentrated in vacuo. The crude product was purified by flash chromatography (CH₂Cl₂/EtOH 95:5) to give 15 mg (30%) of amide 9a of FIG. 4 (FL14) as a white solid. NMR data: see below.

### 3a-(4-Bromophenyl)-1,8b-dihydroxy-8-methoxy-1-oxo-3-phenyl-2,3-3a,8b-tetrahydro-cyclopenta[b]benzofuran-2-carboxamide (FL9, 9b of FIG. 4)

To a saturated solution of 5 mL of ammoniac in MeOH was added ester 6b of FIG. 4 (70 mg, 0.14 mmol) in a sealed tube. The solution was then heated at 100° C. for 36 h. After cooling to rt, the solution was concentrated in vacuo. A pure sample of amide 9b of FIG. 4 (16 mg, 16%) was isolated by RP-HPLC (Symetry shield RP18, 7 μm, 19×300 mm, with a flow rate of 10 mL/min using a 50 min gradient from water (0.1% TFA) to CH₃CN (0.1% TFA)). NMR data: see below.

### Synthesis of FL8 (7b of FIG. 4)

### Methyl 2-(4-bromophenyl)-5-hydroxy-2,5-methano-8-methoxy-10-oxo-2,3,4,5-tetrahydro-1-benzoxepin-4-carboxylate (11 of FIG. 4)

A solution of hydroxyflavone 3b of FIG. 4 (1.0 g, 3 mmol) and dimethyl cinnamamide 10 of FIG. 4 (5.10 g, 30 mmol) in 90 mL of CH₂Cl₂/MeOH (3:1) was degassed with argon for 10 min in a pyrex tube. This mixture was then irradiated (450 W Iwasaki UV lamp) for 25 h at 0° C. under an argon atmosphere. The solution was concentrated in vacuo, purified by flash chromatography (heptane/AcOEt 4:6), heated to reflux in EtOAc (20 mL) for 4 h and concentrated in vacuo to give 560 mg (18%) of adduct 11 of FIG. 4 as a white solid. ¹H NMR (CDCl₃): 2.87 (3H, s), 3.23 (3H, s), 3.80 (3H, m), 3.90 (1H, d, J=7.7 Hz), 4.99 (1H, d, J=7.7 Hz), 6.55 (2H, m), 6.80-7.20 (10H, m). ¹³C NMR (CDCl₃): 36.4, 37.8, 51.4, 55.6, 60.6, 88.3, 97.9, 101.3, 108.1, 118.2, 121.1, 125.1, 127.6, 128.2, 130.0, 130.9, 134.6, 137.5, 152.8, 161.9, 171.9, 191.7.

### Methyl 3a-(4-bromophenyl)-8b-hydroxy-8-methoxy-1-oxo-3-phenyl-2,3-3a,8b-tetrahydro-cyclopenta[b]benzofuran-2-carboxylate

To a solution of aglain 11 of FIG. 4 (150 mg, 0.3 mmol) in MeOH (15 mL) was added a solution of NaOMe in MeOH (0.4 M, 2 mL) at 0° C. The resulting solution was stirred for 20 min at 60° C., cooled to rt, and quenched with saturated NH₄Cl, extracted with AcOEt (15 mL). The organic layer was washed with water (2×20 mL) and brine (20 mL), dried over MgSO₄ and concentrated in vacuo to afford 140 mg of the title β-ketoester as a white solid which was used in the next step without further purification. ¹H NMR (CDCl₃): 2.88 (3H, s), 3.22 (3H, s), 3.83 (4H, m), 4.37 (1H, s), 6.58 (1H, dd, J=2.2, 8.5 Hz), 6.67 (1H, d, J=2.2 Hz), 6.83 (2H, m), 7.10 (3H, m), 7.30 (5H, m). ¹³C NMR (CDCl₃): 36.5, 37.4, 51.6, 55.5, 56.1, 88.3, 96.7, 100.8, 108.4, 115.6, 118.5, 126.1, 127.3, 128.4, 128.5, 128.9, 129.3, 133.0, 138.8, 160.2, 172.3, 203.6.

### 3a-(4-Bromophenyl)-1,8b-dihydroxy-8-methoxy-N,N-dimethyl-1-oxo-3-phenyl-2,3-3a,8b-tetrahydro-cyclopenta[b]benzofuran-2-carboxamide (FL8, 7b of FIG. 4)

Glacial acetic acid (142 mL, 2.49 mmol) was added to a solution of Me₄NBH(OAc)₃ (390 mg, 1.49 mmol) in CH₃CN (15 mL). After stirring for 5 min at room temperature, a solution of ketone 5b of FIG. 4 (130 mg, 0.25 mmol) in CH₃CN (5 mL) was added dropwise. The resulting mixture was stirred for 3 h at rt, successively quenched with saturated aqueous NH₄Cl (20 mL) and a 3 M aqueous solution of sodium/potassium tartrate (10 mL) and stirred for 30 min. The aqueous solution was extracted with CH₂Cl₂ (2×30 mL). The combined organic layers were washed with brine, dried over MgSO₄ and concentrated in vacuo. Purification by HPLC (Symetry shield RP18, 7 μm, 19×300 mm, with a flow rate of 10 mL/min using a 50 min gradient from water (0.1% TFA) to CH₃CN (0.1% TFA)) yielded diol 7b of FIG. 4 (60 mg, 46%) as a white solid. NMR data: see below.

### Methyl 2-(4-bromophenyl)-6,8-dimethoxy-5-hydroxy-2,5-methano-10-oxo-3-(3-fluorophenyl)-2,3,4,5-tetrahydro-1-benzoxepin-4-carboxylate (4d of FIG. 4)

A solution of hydroxyflavone 3a of FIG. 4 (1.0 g, 3.7 mmol) and methyl (E)-3-fluorocinnamate (2.58 g, 16 mmol) in 90 mL of CH₂Cl₂/MeOH (3:1) was degassed with argon for 10 min in a pyrex tube. This mixture was then irradiated (450 W Iwasaki UV lamp) for 40 h at 0° C. under an argon atmosphere. The solution was concentrated in vacuo, purified by flash chromatography (heptane/AcOEt 8:2 to 6:4), heated to reflux in EtOAc (20 mL) for 4 h and concentrated in vacuo to give 225 mg (16%) of adduct 4d of FIG. 4 as a white solid. ¹H NMR (CDCl₃): 3.56 (3H, s), 3.61 (1H, d, J=9.4 Hz), 3.72 (3H, s), 3.81 (3H, s), 4.15 (1H, d, J=9.4 Hz), 6.07 (1H, d, J=2.2 Hz), 6.15 (1H, d, J=2.2 Hz), 6.68 (1H, m), 6.90 (3H, m), 7.23 (2H, d, J=8.7 Hz), 7.51 (2H, d, J=8.7 Hz). ¹³C NMR (CDCl₃): 51.7, 54.3, 55.1, 55.4, 56.0, 88.5, 92.5, 94.9, 98.5, 104.1, 112.6, 114.0, 116.7, 125.6, 127.8, 129.1-129.2 (d, 7.9 Hz), 130.5, 142.5-142.6 (d, 7.5 Hz), 153.9, 158.5, 158.9, 166.1-164.3 (d, 245.0 Hz), 161.3, 171.3, 172.2.

### Methyl 3a-(4-bromophenyl)-6,8-dimethoxy-8b-hydroxy-1-oxo-3-(3-fluorophenyl)-2,3-3a,8b-tetrahydro-cyclopenta[b]benzofuran-2-carboxylate (5d of FIG. 4)

To a solution of aglain 4d of FIG. 4 (500 mg, 0.9 mmol) in MeOH (50 mL) was added a solution of NaOMe in MeOH (0.4 M, 5 mL) at 0° C. The resulting solution was stirred for 20 min at 60° C., cooled to rt, and quenched with saturated NH₄Cl, extracted with AcOEt (40 mL), and the organic layer was washed with water (2×20 mL) and brine (20 mL), dried over MgSO₄ and concentrated in vacuo to afford 370 mg of crude β-ketoester 5d of FIG. 4 as a white solid which was used without further purification. ¹H NMR (CDCl₃): 3.67 (3H, s), 3.79 (3H, s), 3.84 (3H, s), 3.99 (1H, d, J=13.2 Hz), 4.23 (1H, d, J=13.2 Hz), 6.10 (1H, d, J=2.0 Hz), 6.34 (1H, d, J=2.0 Hz), 6.66 (1H, m), 6.92 (2H, d, J=8.6 Hz), 7.10 (3H, m), 7.28 (2H, d, J=8.6 Hz). ¹³C NMR (CDCl₃): 51.8, 53.4, 55.9, 56.0, 56.3, 88.6, 89.4, 90.2, 93.5, 105.7, 114.2, 115.4, 120.7, 128.5, 129.3, 129.9-130.0 (d, 8.1 Hz), 131.2, 132.5, 137.8-137.9 (d, 7.3 Hz), 158.2, 160.6-162.9 (d, 232.9 Hz), 164.1, 165.4, 168.1, 202.7.

### Methyl 3a-(4-bromophenyl)-1,8b-dihydroxy-6,8-dmethoxy-1-oxo-3-(3-fluorophenyl)-2,3-3a,8b-tetrahydro-cyclopenta[b]benzofuran-2-carboxylate (FL17, 6d of FIG. 4)

Glacial acetic acid (520 μL, 4.33 mmol) was added to a solution of Me₄NBH(OAc)₃ (1.42 g, 5.38 mmol) in CH₃CN (80 mL). After stirring for 5 min at room temperature, a solution of ketone 5d of FIG. 4 (500 mg, 0.90 mmol) in CH₃CN (10 mL) was added dropwise. The resulting mixture was stirred for 3 h at rt, successively quenched with saturated aqueous NH₄Cl (30 mL) and a 3 M aqueous solution of sodium/potassium tartrate (20 mL) and stirred for 30 min. The aqueous solution was extracted with CH₂Cl₂ (2×40 mL). The combined organic layers were washed with brine, dried over MgSO₄ and concentrated in vacuo. The crude product was purified by flash chromatographie (CH₂Cl₂/Et₂O 95:5) to give diol 6d of FIG. 4 (265 mg, 53%) as a white solid. ¹H NMR (CDCl₃): 3.56 (1H, s, OH), 3.66 (3H, s), 3.81 (3H, s), 3.8 (4H, m), 4.33 (1H, d, J=14.1 Hz), 4.98 (1H, d, J=6.4 Hz), 6.11 (1H, d, J=2.0 Hz), 6.28 (1H, d, J=2.0 Hz), 6.66 (2H, m), 6.77 (1H, m), 7.03 (1H, m), 7.08 (1H, d, J=8.6 Hz), 7.31 (1H, d, J=8.6 Hz). ¹³C NMR (CDCl₃): 50.5, 52.2, 54.8, 55.8, 79.6, 89.6, 93.0, 93.8, 101.5, 107.3, 113.7-113.9 (d, J=21.3 Hz), 114.9-115.1 (d, J=21.6 Hz), 121.9, 123.2, 129.4-129.5 (d, J=8.8 Hz), 129.5, 130.5, 133.7, 139.4-139.5 (d, J=7.3 Hz), 157.0, 160.6, 161.3-163.7 (d, J=245.4 Hz), 164.3, 170.2.

### 3a-(4-Bromophenyl)-1,8b-dihydroxy-6,8-dimethoxy-1-oxo-3-(3-fluorophenyl)-2,3-3a,8b-tetrahydro-cyclopenta[b]benzofuran-2-carboxamide (FL18)

Ester (50 mg, 0.09 mmol) was added to a saturated solution of ammoniac in methanol (5 mL) in a sealed tube. The solution was then heated at 100° C. for 36 h, cooled to rt, and concentrated in vacuo. The crude product was purified by flash chromatography (AcOEt) to give 7 mg (14%) of amide FL18 as a white solid. NMR data: see below.

### Methyl 2-(4-methoxyphenyl)-6,8-dimethoxy-5-hydroxy-2,5-methano-10-oxo-3-(3-fluorophenyl)-2,3,4,5-tetrahydro-1-benzoxepin-4-carboxylate (4e of FIG. 4)

A solution of hydroxyflavone 3d of FIG. 4 (1.0 g, 3.0 mmol) and methyl (E)-3-fluorocinnamate (3.29 g, 18 mmol) in 90 mL of CH₂Cl₂/MeOH (3:1) was degassed with argon for 10 min in a pyrex tube. This mixture was then irradiated (450 W Iwasaki UV lamp) for 40 h at 0° C. under an argon atmosphere. The solution was concentrated in vacuo, purified by flash chromatography (heptane/AcOEt 7:3 to 6:4), heated to reflux in EtOAc (20 mL) for 4 h and concentrated in vacuo to give 400 mg (26%) of adduct 4e of FIG. 4 as a white solid. ¹H NMR (CDCl₃): 3.57 (3H, s), 3.63 (1H, d, J=9.2 Hz), 3.68 (3H, s), 3.74 (3H, s), 3.82 (3H, s), 4.18 (1H, d, J=9.2 Hz), 6.09 (1H, d, J=2.2 Hz), 6.18 (1H, d, J=2.2 Hz), 6.66 (3H, m), 6.98 (3H, m), 7.58 (2H, d, J=8.9 Hz). ¹³C NMR (CDCl₃): 52.0, 54.3, 55.1, 55.4, 56.0, 62.8, 87.7, 92.8, 94.6, 98.1, 103.6, 112.9, 113.4-113.7 (d, 21.0 Hz), 116.5-116.8 (d, 22.1 Hz), 125.8, 127.7, 129.2-129.3 (d, 7.7 Hz), 130.2, 142.8-142.9 (d, 7.7 Hz), 153.7, 158.6, 158.9, 160.9-164.1 (d, 244.4 Hz), 161.4, 171.3, 171.8.

### Methyl 3a-(4-methoxyphenyl)-6,8-dimethoxy-8b-hydroxy-1-oxo-3-(3-fluorophenyl)-2,3-3a,8b-tetrahydro-cyclopenta[b]benzofuran-2-carboxylate (5^{e} of FIG. 4)

To a solution of aglain 4e of FIG. 4 (300 mg, 0.6 mmol) in MeOH (30 mL) was added a solution of NaOMe in MeOH (0.4 M, 5 mL) at 0° C. The resulting solution was stirred for 20 min at 60° C., cooled to rt, and quenched with saturated NH₄Cl, extracted with AcOEt (3×20 mL), and the organic layer was washed with water (2×20 mL) and brine (20 mL), dried over MgSO₄ and concentrated in vacuo to afford 300 mg of crude β-ketoester 5e of FIG. 4 as a white solid which was used without further purification. ¹H NMR (CDCl₃): 3.63 (3H, s), 3.64 (3H, s), 3.74 (3H, s), 3.79 (3H, s), 3.99 (1H, d, J=13.3 Hz), 4.19 (1H, d, J=13.3 Hz), 6.06 (1H, d, J=1.9 Hz), 6.31 (1H, d, J=1.9 Hz), 6.51 (2H, d, J=8.9 Hz), 6.66 (3H, m), 6.80 (1H, m), 6.93 (2H, d, J=8.9 Hz). ¹³C NMR (CDCl₃): 51.8, 53.1, 55.2, 55.7, 55.8, 56.4, 88.6, 90.0, 93.1, 99.3, 112.3, 113.4, 114.1-114.3 (d, 21.6 Hz), 115.0-115.2 (d, 22.4 Hz), 125.3, 127.9, 128.1, 129.5-129.6 (d, 9.2 Hz), 138.3-138.4 (d, 6.6 Hz), 158.7, 159.5-161.9 (d, 244.7 Hz), 161.0, 163.8, 165.1, 167.2, 202.7.

### Methyl 3a-(4-methoxyphenyl)-1,8b-dihydroxy-6,8-dimethoxy-1-oxo-3-(3-fluorophenyl)-2,3-3a,8b-tetrahydro-cyclopenta[b]benzofuran-2-carboxylate (FL16, 6^{e} of FIG. 4).

Glacial acetic acid (110 μL, 2.0 mmol) was added to a solution of Me₄NBH(OAc)₃ (310 mg, 1.18 mmol) in CH₃CN (20 mL). After stirring for 5 min at room temperature, a solution of ketone 5e of FIG. 4 (100 mg, 0.20 mmol) in CH₃CN (5 mL) was added dropwise. The resulting mixture was stirred for 3 h at rt, successively quenched with saturated aqueous NH₄Cl (20 mL) and a 3 M aqueous solution of sodium/potassium tartrate (10 mL) and stirred for 30 min. The aqueous solution was extracted with CH₂Cl₂ (2×20 mL). The combined organic layers were washed with brine, dried over MgSO₄ and concentrated in vacuo. The crude product was purified by flash chromatographie (CH₂Cl₂/Et₂O 95:5) to give diol 6 (FL) (85 mg, 85%) as a white solid. NMR data: see below.

### 3a-(4-methoxyphenyl)-8b-hydroxy-6,8-dimethoxy-3-(3-fluorophenyl)-2,3-3a,8b-tetrahydro-cyclopenta[b]benzofuran-1-one

Lithium chloride (13 mg, 0.3 mmol) and water (11 μL, 0.6 mmol) were added to a solution of keto-ester 5e of FIG. 4 (100 mg, 0.2 mmol) in DMSO (2 mL). The mixture was stirred at 100° C. for 12 h, cooled to room temperature, diluted with 10 mL of water and extracted with AcOEt (3×10 mL). The combined organic layers were washed with brine, dried over MgSO₄ and concentrated to give 75 mg (84%) of the title ketone as a white solid which was used without further purification. ¹H NMR (CDCl₃): 2.95 (2H, m), 3.67 (3H, m), 3.78 (3H, s), 3.81 (4H, m), 6.07 (1H, d, J=1.9 Hz), 6.31 (1H, d, J=1.9 Hz), 6.65 (2H, d, J=8.8 Hz), 6.75 (3H, m), 6.93 (2H, d, J=8.8 Hz), 7.10 (1H, m). ¹³C NMR (CDCl₃): 41.0, 48.4, 55.3, 55.7, 55.9, 88.9, 89.9, 92.9, 101.2, 106.5, 113.4, 113.7-113.9 (d, J=26.9 Hz), 115.1-115.3 (d, J=22.0 Hz), 123.6, 125.7, 128.2, 129.4-129.5 (d, J=7.7 Hz), 140.1-140.2 (d, J=7.3 Hz), 158.6, 159.0, 161.2, 161.4-163.8 (d, J=245.0 Hz), 164.9, 210.2.

### 3a-(4-methoxyphenyl)-6,8-dimethoxy-3-(3-fluorophenyl)-1,2,3,3a-tetrahydro-cyclopenta[b]benzofuran-1,8b(1H)-diol (FL15)

Glacial acetic acid (90 μL, 1.55 mmol) was added to a solution of Me₄NBH(OAc)₃ (245 mg, 0.93 mmol) in CH₃CN (8 mL). After stirring for 5 min at room temperature, a solution of the previous compound (70 mg, 1.55 mmol) in CH₃CN (2 mL) was added dropwise. The resulting mixture was stirred for 3 h at rt, successively quenched with saturated aqueous NH₄Cl (15 mL) and a 3 M aqueous solution of sodium/potassium tartrate (3 mL) and stirred for 30 min. The aqueous solution was extracted with AcOEt (2×30 mL). The combined organic layers were washed with brine, dried over MgSO₄ and concentrated in vacuo. The crude product was purified by flash chromatographie (CH₂Cl₂/Et₂O 95:5) to give diol FL15 (35 mg, 50%) as a white solid. NMR data: see below.

### Synthesis of FL19, FL20 and FL21

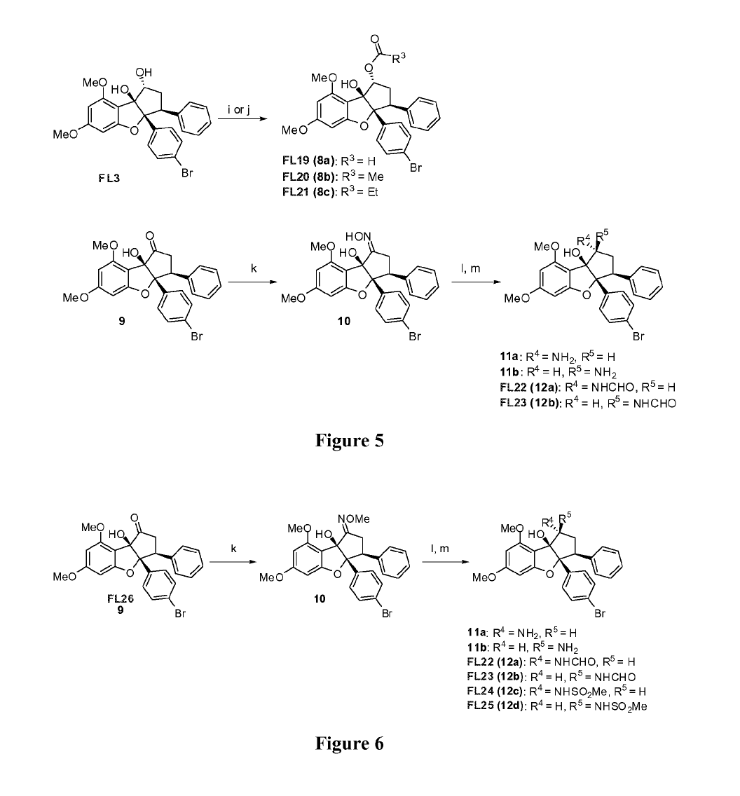

3a-(4-Bromophenyl)-6,8-dimethoxy-3-phenyl-1,2,3,3a-tetrahydro-cyclopenta[b]benzofuran-1,8b(1H)-diol 1-formate (FL19, 8a of FIG. 5).

To a solution of FL3 (50 mg, 0.10 mmol) in anhydrous CH₂Cl₂ (1 mL) was added successively DCC (24 mg, 0.11 mmol) and DMAP (1 mg, 0.01 mmol). After 5 min of stirring, formic acid (4 μL, 0.11 mmol) was added slowly at 0° C. The solution was stirred 36 h at rt. Then, the reaction was diluted with pentane and filtered. The solid was washed with of pentane (2×5 mL) and the filtrate was concentrated in vacuo. The crude product was purified by flash chromatography (CH₂Cl₂) to give 15 mg of formic ester 8a of FIG. 5 (FL19). NMR data: see below.

### 3a-(4-Bromophenyl)-6,8-dimethoxy-3-phenyl-1,2,3,3a-tetrahydro-cyclopenta[b]benzofuran-1,8b(1H)-diol 1-acetate (FL20, 8b of FIG. 5)

To a solution of FL3 (50 mg, 0.10 mmol) in anhydrous pyridine (1 mL), was added DMAP (3 mg, 0.03 mmol) and acetic anhydride (67 μL, 0.50 mmol). The solution was sonicated until complete solubilisation and stirred at rt for 6 h. The mixture was then concentrated in vacuo and purified by flash chromatography (CH₂Cl₂) to give 42 mg (84%) of ester 8b of FIG. 5 (FL20). NMR data: see below.

### 3a-(4-Bromophenyl)-6,8-dimethoxy-3-phenyl-1,2,3,3a-tetrahydro-cyclopenta[b]benzofuran-1,8b(1H)-diol 1-propionate (FL21, 8c of FIG. 5)

To a solution of FL3 (50 mg, 0.10 mmol) in anhydrous pyridine (1 mL), was added DMAP (3 mg, 0.03 mmol) and propionic anhydride (67 μL, 0.50 mmol). The solution was sonicated until complete solubilisation and stirred at rt for 6 h. The mixture was then concentrated in vacuo and purified by flash chromatography (CH₂Cl₂) to give 49 mg (98%) of ester 8c of FIG. 5 (FL21). NMR data: see below.

### Synthesis of FL22 and FL23 (12a and 12b of FIG. 5)

### 3a-(4-Bromophenyl)-6,8-dimethoxy-8b-hydroxy-3-phenyl-2,3-3a,8b-tetrahydro-cyclopenta[b]benzofuran-1-one oxime (10 of FIG. 5)

A solution of ketone 9 of FIG. 5 (375 mg, 0.78 mmol) and hydroxylammonium chloride (270 mg, 3.89 mmol) in 10 mL of pyridine-EtOH (1:1) was stirred at 70° C. for 4 hours. After concentration in vacuo, the residue was dissolved in ethyl acetate. The organic phase was successively washed with 1 N HCl, saturated Na₂CO₃ solution and brine, then dried over magnesium sulfate, filtered, concentrated and purified on silica gel 60 (ether) to yield 330 mg of ketone 10 of FIG. 5 as a white solid. ¹H NMR (400 MHz, DMSO-d₆): 11.15 (s, 1H), 7.34 (d, J=8.5 Hz, 2H), 7.12-7.06 (m, 3H), 6.99-6.97 (m, 4H), 6.39 (d, J=2 Hz, 1H), 6.18 (d, J=2 Hz, 1H), 5.35 (s, 1H), 3.80 (s, 3H), 3.75 (s, 3H), 3.54 (dd, J=12.4 Hz and J=9.8 Hz, 1H), 3.07-2.90 (m, 2H). ¹³C NMR (100 MHz, DMSO-d₆): 163.2, 159.5, 158.4 (X2), 137.9, 134.8, 130.0, 128.8, 127.7, 127.6, 126.5, 120.0, 109.2, 101.3, 92.7, 89.6, 86.8, 55.5, 55.4, 48.8, 29.0. LC-MS (ESI): exact mass (C₂₅H₂₂BrNO₅)=495.07. found: 496.0 [M+H].

### 3a-(4-Bromophenyl)-6,8-dimethoxy-8b-hydroxy-3-phenyl-1,2,3,3a-tetrahydro-cyclopenta[b]benzofuran-1,8b(1H)-formamide (FL22 and FL23, 12a and 12b)

A solution of oxime 10 of FIG. 5 (280 mg, 0.564 mmol) in THF (3 mL) is added to a solution of LiAlH₄ (65 mg, 1.69 mmol) in THF (5 mL). The mixture was stirred for 3 h at 45° C., cooled to 0° C., diluted with EtOAc (10 mL), and quenched with the dropwise addition of 1 M NaOH (10 mL), stirred for 10 min, extracted with EtOAc, washed with brine, dried over MgSO4 and concentrated to dryness. Purification by chromatography on silica gel 60 (Et₂O/MeOH 95:5 to 0:100) yielded 100 mg of the expected primary as a white solid. One drop of acetic acid was added to a solution of this amine (40 mg, 0.083 mmol) in ethyl formate (1.1 mL) and THF (2 mL). The mixture was stirred for 2 h at reflux overnight, concentred and purified by chromatography (Et₂O) to yield formamides 12a of FIG. 5 (FL22, 10 mg) and 12b (FL23, 23 mg). These compounds were further purified by HPLC (Symetry shield RP18, 7 μm, 19×300 mm, with a flow rate of 10 mL/min using a 50 min gradient from water (0.1% TFA) to CH₃CN (0.1% TFA) to give pure samples of formamides 12a of FIG. 5 (Rt=25.5 min) and 12b of FIG. 5 (Rt=28 min).

### Alternative Synthesis of FL22 and FL23 (12a and 12b of FIG. 6) and Synthesis of FL24 and FL25 (12c and 12d of FIG. 6)

### 3a-4-Bromophenyl)-6,8-dimethoxy-8b-hydroxy-3-phenyl-2,3-3a,8b-tetrahydro-cyclopenta[b]benzofuran-1-(O-methyloxime) (10 of FIG. 6).

A solution of ketone 9 of FIG. 6 (500 mg, 1.04 mmol) and O-methylhydroxylamine hydrochloride (430 mg, 5.2 mmol) in 40 mL of pyridine/EtOH (1:1) was stirred for 3 hours at 70° C. After concentration in vacuo, the residue was dissolved in EtOAc. The organic phase was successively washed with 1 N HCl, aq Na₂CO₃ and brine, dried over MgSO₄, filtered and concentrated to quantitatively give 538 mg of oxime 10 of FIG. 6 as a slightly yellow solid.

### 3a-(4-Bromophenyl)-6,8-dimethoxy-8b-hydroxy-3-phenyl-1,2,3,3a-tetrahydro-cyclopenta[b]benzofuran-1,8b(1H)-formamide (FL22 and FL23, 12a and 12b of FIG. 6)

Oxime 10 of FIG. 6 (538 mg, 1.04 mmol) was dissolved in a solution of BH₃ (1 M, 20 mL) in THF at 0° C. The mixture was stirred under reflux for 4 hours, and stirred overnight at rt. The reaction was quenched by a dropwise addition of 3 M NaOH (30 mL). The aqueous phase was extracted twice with CH₂Cl₂ and the organic phase was successively washed with water and brine, dried over MgSO₄ and concentrated to dryness. Purification by chromatography on silica gel (Et₂O/MeOH 70:30) yielded 221 mg of a mixture of amines 11a and 11b of FIG. 6 as a white solid (44%). One drop of acetic acid was added to a solution of this mixture of amines 11a and 11b of FIG. 6 (130 mg, 0.269 mmol) in ethyl formate (0.35 mL, 4.31 mmol) and THF (6 mL). The mixture was stirred at reflux overnight, concentrated and purified several times by chromatography on silica gel (Et₂O/EtOAc, 60:40) to yield formamides 12a of FIG. 6 (FL22, 11 mg) and 12b of FIG. 6 (FL23, 60 mg).

### 3a-(4-Bromophenyl)-6,8-dimethoxy-8b-hydroxy-3-phenyl-1,2,3,3a-tetrahydro-cyclopenta[b]benzofuran-1,8b(1H)-methanesulfonamide (FL24 and FL25, 12c and 12d)

N-methylmorpholine (114 μL, 10.4 mmol) was added at 0° C. to a solution of amines 11a and 11b of FIG. 6 (100 mg) in CH₂Cl₂ (2 mL). After 10 min at 0° C., mesyl chloride (64 μL, 0.82 mmol) was added dropwise and the solution was then stirred for 4 h at rt. The mixture was quenched with 5 mL of HCl 1 N, and extracted with EtOAc (2×10 mL). The organic layer was washed with a solution of Na₂CO₃ (10 mL), brine (10 mL), dried over MgSO₄, and concentrated under reduced pressure. The crude product was purified by flash chromatography on silica gel (CH₂Cl₂/Et₂O, 95:5) to give 12c of FIG. 6 (FL24, 5 mg, 4%) and 12d of FIG. 6 (FL25, 70 mg, 60%).

| | |
|---|---|
| 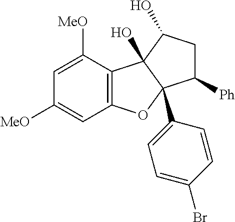 FL3 | NMR ¹H (300 MHz, CDCl₃): 1.89 (1H, br s), 2.14 (1H, dd, J = 7.0, 13.3 Hz), 2.67 (1H, ddd, J = 6.4, 13.9, 13.9 Hz), 3.27 (1H, br s), 3.83 (3H, s), 3.86 (3H, s), 3.99 (1H, dd, J = 6.5, 14.0 Hz), 4.77 (1H, d, J = 6.0 Hz), 6.1 (1H, d, J = 1.8 Hz), 6.28 (1H, d, J = 1.8 Hz), 6.95-6.98 (2H, m), 7.05-7.12 (5H, m), 7.23-7.26 (2H, m). NMR ¹³C (75 MHz, CDCl₃): 36.1; 53.2; 55.6; 55.7; 79.0; 89.4; 92.5; 94.8; 103.1; 107.4; 121.4; 126.4; 127.7; 127.9; 129.4; 130.2; 134.1; 138.1; 156.8; 160.6; 163.9. IR (thin film): 3486, 2942, 2841, 1624, 1598, 1147 cm⁻¹. HR-MS calcd for C₂₅H₂₃Br₁K₁O₆: 521.0366; found: 521.0360 (−2.85 ppm). |
| 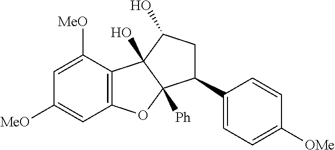 FL4 | NMR ¹H (300 MHz, CDCl₃): 1.82 (1H, br s), 2.26 (1H, s), 2.83 (1H, dd, J = 6.9, 12.6 Hz), 3.44 (1H, br s), 3.80 (3H, s), 3.94 (3H, s), 4.00 (3H, s), 4.09 (1H, m), 4.92 (1H, d, J = 5.2 Hz), 6.25 (1H, s), 6.39 (1H, s), 6.72 (2H, m), 6.96 (2H, m), 7.24-7.36 (5H, m). NMR ¹³C (CDCl₃): 36.7, 52.8, 55.1, 55.7, 55.8, 79.2, 89.4, 92.5, 94.9, 103.6, 107.9, 113.0, 127.0, 127.2, 127.7, 129.0, 130.6, 134.9, 157.0, 158.0, 160.9, 163.9. IR (thin film): 3504, 2942, 2838, 2359, 2597, 1597, 1513, 1146. HR-MS calcd for C₂₆H₂₆Na₁O₆: 457.1627, found: 457.1622. |
| 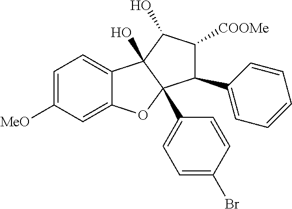 FL5 | NMR ¹H (300 MHz, CDCl₃): 3.67 (3H, s), 3.87 (3H, s), 3.92 (1H, dd, J = 14, 6.2 Hz), 4.45 (1H, d, J = 14 Hz), 4.83 (1H, d, J = 6.2 Hz), 6.60 (2H, m), 6.90-7.15 (7H, m), 7.23 (2H, d, J = 8.6 Hz), 7.33 (1H, d, J = 8.1 Hz). NMR ¹³C (75 MHz, CDCl₃): 51.1, 52.8, 56.2, 79.5, 93.6, 97.3, 101.8, 109.1, 119.1, 122.2, 127.5, 128.3, 128.7, 129.8, 131.0, 134.4, 137.0, 160.9, 163.6, 172.0. |
| 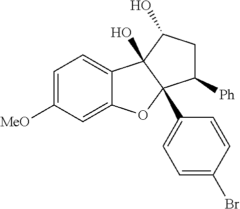 FL6 | NMR ¹H (300 MHz, CDCl₃): 2.36 (1H, ddd, J = 2.8, 7.0, 13.5 Hz), 2.79 (1H, dt, J = 5.3, 13.4 Hz), 3.88 (3H, s), 4.27 (1H, dd, J = 6.9, 13.4 Hz), 4.71 (1H, dd, J = 2.7, 5.1 Hz), 6.64 (2H, m), 6.90-7.30 (10H, m). NMR ¹³C (75 MHz, CDCl₃): 36.9, 53.6, 55.7, 78.4, 94.1, 96.7, 102.8, 108.4, 118.6, 121.6, 126.3, 126.5, 128.0, 128.2, 129.3, 130.5, 134.8, 138.7, 160.8, 163.0. |
| 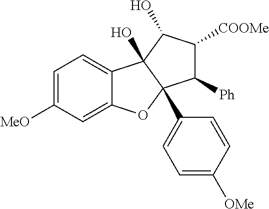 FL7 | NMR ¹H (300 MHz, CDCl₃): 2.42(1H, d, J = 5.4 Hz), 3.69 (3H, s), 3.70 (3H, s), 3.83 (3H, s), 3.93 (1H, dd, J = 5.4, 13.4 Hz), 4.43 (1H, d, J = 13.4 Hz), 4.83 (1H, t, J = 5.4 Hz), 6.60 (4H, m), 6.90-7.15 (7H, m), 7.31 (1H, d, J = 8.3 Hz). NMR ¹³C (75 MHz, CDCl₃): 51.1, 52.6, 55.4, 55.7, 56.0, 79.3, 93.0, 97.1, 101.9, 108.6, 113.2, 119.5, 126.9, 127, 127.5, 128.2, 128.4, 129.2, 137.4, 159.0, 160.9, 163.1, 172.0. |
| 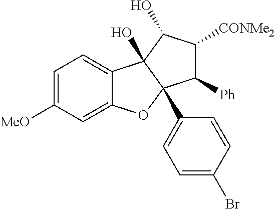 FL8 | NMR ¹H (300 MHz, CDCl₃): 2.86 (3H, s), 3.19 (3H, s), 4.80 (3H, s), 4.06 (1H, dd, J = 7.0, 13.4 Hz), 4.40 (1H, d, J = 13.3 Hz), 4.77 (1H, d, J = 7.0 Hz), 6.55 (2H, m), 6.77 (2H, m), 7.02 (5H, m, H2), 7.24 (2H, m), 7.37 (1H, d, J = 7.9 Hz). ¹³C NMR (CDCl₃): 36.2, 37.7, 46.7, 55.8, 57.1, 78.5, 92.2, 96.8, 101.0, 108.6, 119.7, 121.8, 127.1, 128.1, 128.2, 129.6, 130.7, 134.4, 136.8, 161.1, 163.8, 172.2. |
| 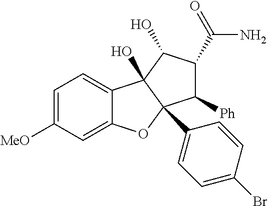 FL9 | NMR ¹H (300 MHz, CDCl₃): 3.81 (3H, s, H19), 3.94 (1H, dd, J = 5.7, 13.5 Hz), 4.45 (1H, d, J = 13.5 Hz), 4.85 (1H, d, J = 5.6 Hz), 6.59 (2H, m), 6.90-7.30 (10H, m). NMR ¹³C (75 MHz, CDCl₃): 53.6, 55.7, 78.9, 93.1, 97.3, 101.4, 108.7, 118.8, 121.8, 127.2, 128.0, 128.6, 129.4, 130.5, 134.0, 136.6, 160.3, 163.0, 174.8. |
| 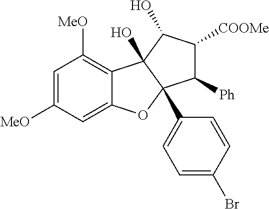 FL10 | NMR ¹H (300 MHz, CDCl₃): 3.64 (3H, s), 3.82 (3H, s), 3.86 (3H, s), 3.92 (1H, dd, J = 14.1, 6.6 Hz), 4.33 (1H, d, J = 14.1 Hz), 5.01 (1H, d, J = 6.6 Hz), 6.12 (1H, d, J = 2.0 Hz), 6.27 (1H, d, J = 2.0 Hz), 6.87 (2H, m), 7.07 (5H, m), 7.25 (2H, d, J = 8.3 Hz). NMR ¹³C (75 MHz, CDCl₃): 50.4, 52.1, 55.1, 55.8, 60.5, 79.7, 89.6, 92.8, 93.8, 101.7, 107.6, 121.7, 126.9, 127.8, 128.0, 129.6, 130.3, 134.1, 136.7, 157.1, 160.7, 164.3, 170.5. |
| 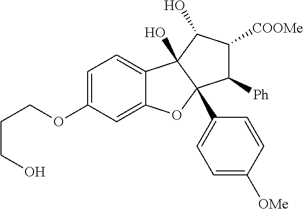 FL11 | NMR ¹H (300 MHz, CDCl₃): 2.12 (2H, quint, J = 6.1 Hz); 3.60 (3H, s); 3.78 (3H, s); 4.21 (4H, m); 3.93 (2H, t, J = 5.7 Hz); 5.06 (1H, d, J = 7.3 Hz); 6.71 (6H, m); 7.11 (5H, m); 7.47 (1H, d, J = 8.9 Hz). NMR ¹³C (75 MHz, CDCl₃): 31.9, 49.5, 52.0, 55.1, 55.4, 60.4, 65.9, 78.9, 96.9, 97.5, 98.8, 108.8, 112.6, 127.2, 127.3, 127.9, 128.0, 128.1, 129.2, 136.3, 158.6, 161.6, 171.0. |
| 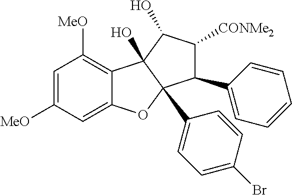 FL12 | ¹H NMR (CDCl₃): 2.92 (3H, s), 3.30 (3H, s), 3.82 (3H, s), 3.83 (3H, s), 4.03 (1H, dd, J = 6.3, 13.5 Hz), 4.57 (1H, d, J = 13.5 Hz), 4.89 (1H, d, J = 6.3 Hz), 6.09 (1H, d, J = 1.8 Hz), 6.26 (1H, d, J = 1.8 Hz), 6.83 (2H, m), 7.03 (5H, m), 7.24 (2H, d, J = 8.6 Hz). ¹³C NMR (CDCl₃): 36.1, 37.3, 47.7, 55.9, 56.3, 78.9, 89.5, 92.9, 94.4, 101.7, 107.5, 121.7, 126.8, 127.9, 128.1, 129.6, 130.5, 134.7, 137.4, 157.4, 161.6, 164.3, 169.6. |
| 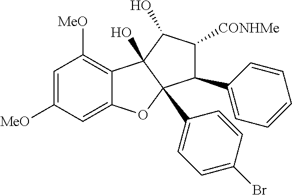 FL13 | ¹H NMR (CDCl₃): 2.76 (3H, d, J = 4.9 Hz), 3.78 (1H, dd, J = 5.1, 14.1 Hz), 3.89 (3H, s), 3.90 (3H, s), 4.44 (1H, d, J = 14.1 Hz), 4.82 (1H, d, J = 5.1 Hz), 6.19 (1H, d, J = 2.0 Hz), 6.33 (1H, d, J = 2.0 Hz), 7.08 (2H, m), 7.15 (5H, m), 7.28 (2H, d, J = 8.8 Hz). ¹³C NMR (CDCl₃): 26.2, 51.7, 55.8, 56.1, 79.4, 89.1, 92.3, 93.7, 101.8, 106.8, 121.3, 126.8, 128.0, 128.2, 129.6, 130.1, 134.9, 136.7, 157.6, 161.1, 164.2, 170.6. |
| 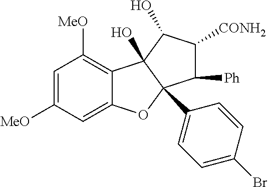 FL14 | ¹H NMR (CDCl₃): 3.76 (1H, dd, J = 5.4, 14.1 Hz), 3.80 (3H, s), 3.81 (3H, s), 4.28 (1H, d, J = 14.1 Hz), 4.88 (1H, d, J = 5.4 Hz), 6.09 (1H, d, J = 1.8 Hz), 6.24 (1H, d, J = 1.8 Hz), 6.98 (2H, m), 7.06 (4H, m), 7.19 (2H, d, J = 8.7 Hz), 7.43 (1H, d, J = 8.7 Hz). ¹³C NMR (CDCl₃): 51.8, 55.9, 56.3, 79.3, 89.4, 92.8, 93.8, 101.9, 106.9, 121.8, 127.3, 128.3, 128.4, 129.7, 130.4, 134.3, 136.2, 157.5, 161.1, 164.4, 172.9. |
| 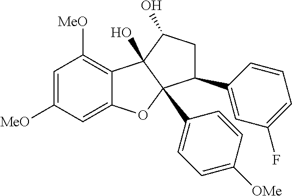 FL15 | ¹H NMR (CDCl₃): 2.15 (1H, dd, J = 6.4, 13.6 Hz), 2.67 (1H, dt, J = 6.4, 13.7 Hz), 3.68 (3H, s), 3.81 (3H, s), 3.86 (3H, s), 3.95 (1H, dd, J = 6.4, 13.9 Hz), 4.76 (1H, d, J = 6.4 Hz), 6.11 (1H, d, J = 2.0 Hz), 6.26 (1H, d, J = 2.0 Hz), 6.66 (2H, d, J = 8.9 Hz), 6.73 (3H, m), 7.03 (1H, m), 7.10 (2H, d, J = 8.9 Hz). ¹³C NMR (CDCl₃): 36.5, 53.1, 55.3, 55.9, 56.0, 79.1, 89.6, 92.7, 95.0, 103.5, 107.8, 113.0, 113.2-113.4 (d, J = 20.9 Hz), 115.1-115.3 (d, J = 21.6 Hz), 123.9, 126.7, 129.0, 129.1-129.2 (d, J = 8.1 Hz), 141.6-141.7 (d, J = 7.3 Hz), 157.2, 158.9, 161.1, 161.4-163.8 (d, J = 244.3 Hz), 164.2. |
| 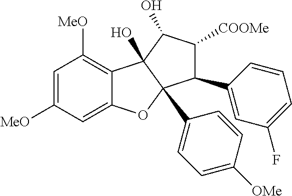 FL16 | ¹H NMR (CDCl₃): 3.63 (3H, s), 3.66 (3H, s), 3.79 (3H, s), 3.82 (4H, m), 4.26 (1H, d, J = 14.0 Hz), 4.99 (1H, d, J = 6.6 Hz), 6.09 (1H, d, J = 1.9 Hz), 6.25 (1H, d, J = 1.9 Hz), 6.6 (4H, m), 6.73 (1H, m), 6.99 (1H, m), 7.08 (1H, d, J = 8.9 Hz). ¹³C NMR (CDCl₃): 50.6, 52.2, 54.9, 55.3, 55.8, 55.9, 79.7, 89.7, 92.9, 93.8, 101.8, 107.8, 113.5-113.7 (d, J = 20.9 Hz), 115.0-115.2 (d, J = 22.0 Hz), 123.5, 126.3, 129.0, 129.2, 129.2-129.3 (d, J = 8.4 Hz), 139.9-140.0 (d, J = 7.3 Hz), 157.1, 159.0, 160.9, 161.3-163.8 (d, J = 244.7 Hz), 164.3, 170.4. |
| 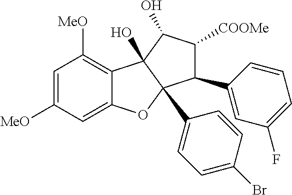 FL17 | ¹H NMR (CDCl₃): 3.56 (1H, s, OH), 3.66 (3H, s), 3.81 (3H, s), 3.8 (4H, m), 4.33 (1H, d, J = 14.1 Hz), 4.98 (1H, d, J = 6.4 Hz), 6.11 (1H, d, J = 2.0 Hz), 6.28 (1H, d, J = 2.0 Hz), 6.66 (2H, m), 6.77 (1H, m), 7.03 (1H, m), 7.08 (1H, d, J = 8.6 Hz), 7.31 (1H, d, J = 8.6 Hz). ¹³C NMR (CDCl₃): 50.5, 52.2, 54.8, 55.8, 79.6, 89.6, 93.0, 93.8, 101.5, 107.3, 113.7-113.9 (d, J = 21.3 Hz), 114.9-115.1 (d, J = 21.6 Hz), 121.9, 123.2, 129.4-129.5 (d, J = 8.8 Hz), 129.5, 130.5, 133.7, 139.4-139.5 (d, J = 7.3 Hz), 157.0, 160.6, 161.3-163.7 (d, J = 245.4 Hz), 164.3, 170.2. |
| 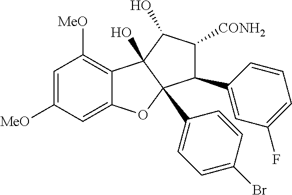 FL18 | ¹H NMR (CDCl₃): 3.74 (1H, dd, J = 5.5, 14.1 Hz), 3.79 (3H, s), 3.80 (3H, s), 4.29 (1H, d, J = 14.1 Hz), 4.75 (1H, d, J = 5.5 Hz), 6.08 (1H, d, J = 1.9 Hz), 6.23 (1H, d, J = 1.9 Hz), 6.72 (3H, m), 7.00 (1H, m), 7.07 (2H, d, J = 8.7 Hz), 7.19 (1H, d, J = 8.7 Hz). ¹³C NMR (CDCl₃): 50.9, 51.6, 55.5, 55.6, 78.9, 88.9, 92.4, 93.7, 101.2, 107.6, 113.2-113.4 (d, J = 20.9 Hz), 115.1-115.3 (d, J = 22.0 Hz), 123.6, 129.2, 129.3, 129.4- 129.5 (d, J = 8.2 Hz), 130.0, 133.2, 140.2-140.3 (d, J = 7.4 Hz), 157.0, 159.3, 160.7, 161.3-163.8 (d, J = 244.9 Hz), 164.0, 171.7. |
| 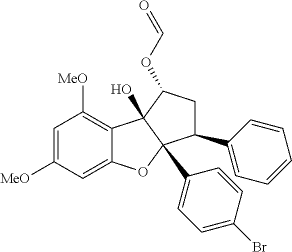 FL19 | ¹H NMR (CDCl₃): 2.34 (1H, ddd, J = 13.6, 6.4, 2.1 Hz), 2.87 (1H, td, J = 13.2, 4.9 Hz), 3.76 (3H, s), 3.82 (3H, s), 4.09 (1H, dd, J = 13.6, 6.1 Hz), 5.89 (1H, dd, J = 4.9, 2.1 Hz), 6.05 (1H, d, J = 1.9 Hz), 6.22 (1H, d, J = 1.9 Hz), 7.06 (7H, m), 7.21 (1H, d, J = 8.5 Hz).. ¹³C NMR (CDCl₃): 35.8, 54.1, 55.6, 55.8, 79.5, 88.7, 92.4, 93.7, 103.0, 106.4, 121.8, 126.9, 128.1, 128.2, 129.5, 130.5, 134.7, 138.0, 158.0, 160.0, 160.9, 164.3. |
| 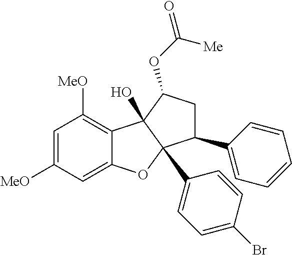 FL20 | ¹H NMR (CDCl₃): 1.85 (3H, s), 2.28 (1H, ddd, J = 13.5, 6.5, 2.3 Hz), 2.38 (s, OH), 2.80 (1H, td, J = 13.5, 5.4 Hz), 3.75 (3H, s), 3.82 (3H, s), 4.06 (1H, dd, J = 13.0, 6.5 Hz), 5.83 (1H, dd, J = 5.4, 2.3 Hz), 6.04 (1H, d, J = 1.9 Hz), 6.21 (1H, d, J = 1.9 Hz), 7.08 (7H, m), 7.20 (1H, d, J = 8.5 Hz). ¹³C NMR (CDCl₃): 21.1, 35.5, 54.1, 55.6, 55.8, 79.7, 88.5, 92.1, 93.7, 103.0, 106.9, 121.6, 126.7, 128.1, 128.2, 129.6, 130.4, 134.9, 138.3, 158.1, 160.8, 164.0, 170.1. |
| 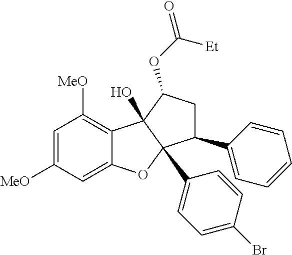 FL21 | ¹H NMR (CDCl₃): 0.94 (3H, t, J = 7.5 Hz), 2.10 (2H, m), 2.30 (2H, m), 2.82 (1H, td, J = 13.7, 5.1 Hz), 3.73 (3H, s), 3.82 (3H, s), 4.10 (1H, dd, J = 13.7, 6.1 Hz), 5.81 (1H, dd, J = 5.1, 2.0 Hz), 6.02 (1H, d, J = 2.0 Hz), 6.21 (1H, d, J = 2.0 Hz), 6.66 (2H, m), 7.02 (1H, d, J = 8.8 Hz), 7.09 (5H, m), 7.19 (1H, d, J = 8.8 Hz). ¹³C NMR (CDCl₃): 9.1, 27.9, 35.7, 54.1, 55.5, 55.8, 79.3, 88.5, 92.0, 93.7, 103.0, 106.7, 121.6, 126.7, 128.1, 128.2, 129.5, 130.4, 135.0, 138.3, 158.0, 160.9, 164.0, 173.2. |
| 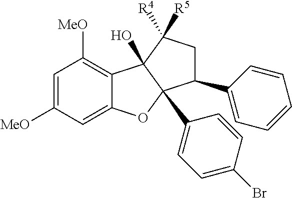 FL22 (12a): R⁴ = NHCHO, R⁵ = H FL23 (12b): R⁴ = H, R⁵ = NHCHO | FL22: ¹H NMR (400 MHz, CDCl₃): (s, 1H), 7.18 (d, J = 8.8 Hz, 2H), 7.13-7.02 (m, 5H), 6.99 (d, J = 8.7 Hz, 2H), 6.22 (d, J = 2 Hz, 1H), 6.09 (d, J = 2 Hz, 1H), 5.03 (dd, J = 7.2 Hz and J = 14.2 Hz, 1H), 3.88-3.83 (m, 1H), 3.83 (s, 3H), 3.82 (s, 3H), 2.87-2.80 (m, 1H), 2.23-2.16 (m, 1H). FL23: ¹H NMR (400 MHz, CDCl₃): δ = 8.23 (s, 1H), 7.15 (d, J = 8.8 Hz, 2H), 7.02-6.92 (m, 7H), 6.15 (d, J = 2 Hz, 1H), 5.99 (d, J = 2 Hz, 1H), 4.66 (dd, J = 7.2 Hz and J = 11.2 Hz, 1H), 3.75 (s, 3H), 3.71 (s, 3H), 3.55 (dd, J = 5.6 Hz and J = 14.4 Hz, 1H), 2.75-2.69 (m, 1H), 2.25- 2.19 (m, 1H). LC-MS (ESI): exact mass (C₂₆H₂₄BrNO₅) = 509.08; found: 510.0 [M + H⁺]. |
| 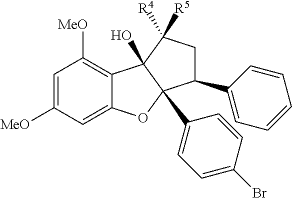 FL24 (12c): R⁴ = NHSO₂Me, R⁵ = H FL25 (12d): R⁴ = H, R⁵ = NHSO₂Me | FL24: ¹H NMR (400 MHz, CD₃OD/CDCl₃): 7.23 (d, J = 8.5 Hz, 2H), 7.10-6.96 (m, 7H), 6.22 (d, J = 1.9 Hz, 1H), 6.08 (d, J = 1.9 Hz, 1H), 5.65 (d, J = 4.4 Hz, 1H), 4.34 (m, 1H), 3.81 (s, 6H), 3.55 (d, J = 14.7, 6.0, 1H), 3.06 (s, 3H), 2.72 (m, 1H), 2.45 (m, 1H). ¹³C NMR (100 MHz, CD₃OD/CDCl₃): 164.4, 159.9, 157.5, 137.3, 134.2, 130.7, 130.1, 128.1, 126.9, 121.8, 110.0, 102.2, 92.8, 89.3, 87.1, 58.0, 55.9, 55.6, 51.3, 40.7, 37.8. LC-MS (ESI): exact mass (C₂₆H₂₆BrNO₆S) = 559.07; found: 560.0 [M + H⁺]. FL25: ¹H NMR (400 MHz, CD₃OD/CDCl₃): 7.18 (d, J = 8.5 Hz, 2H), 7.09-6.93 (m, 7H), 6.17 (d, J = 2 Hz, 1H), 6.04 (d, J = 2 Hz, 1H), 4.47 (m, 1H), 4.32 (m, 1H), 3.96 (m, 1H), 3.79 (s, 3H), 3.78 (s, 3H), 2.93 (s, 3H), 2.47 (m, 1H). LC-MS (ESI): exact mass (C₂₆H₂₆BrNO₆S) = 559.07; found: 560.0 [M + H⁺]. |

### Example 4

### Effect of Rocaglaol and its Derivatives on Human Cancer Cell Viability and on Doxorubicin Cardiotoxicity

### Synthesis of Racemic Rocaglaol and its Derivatives

Racemic rocaglaol (FL1) and its derivatives (FL2-4) were synthesised by the approach developed by Dobler and collaborators (Dobler et al., 2001) (FIGS. 2 and 3). The synthesis of FL3 and FL5 is detailed in examples 1 and 2. The synthesis of FL6 to FL23 is detailed in example 3. Rocaglaol derivatives FL5-11 were synthesized according to the strategy of Porco and collaborators (Gerard et al., 2004) (FIG. 3). Rocaglaol derivatives FL5 to FL10 and FL12 to FL18 were synthesized according to the strategy of FIG. 4. Rocaglaol derivatives FL19 to FL25 were synthesized according to the strategy of FIGS. 5 and 6.

### Materials

KRIBB3 and doxorubicin were purchased from Sigma-Aldrich (Sigma-Aldrich, Saint-Quentin Fallavier, France). Rabbit polyclonal anti-phospho-HSP27 (ser 82) antibody, rabbit polyclonal anti-HSP27 antibody and horseradish peroxidase-conjugated secondary goat anti-rabbit IgG were purchased from Santa Cruz (Santa Cruz Biotechnology, Santa Cruz, Calif., USA). Enzyme-linked chemiluminescence were purchased from Amersham (Amersham Biosciences, Indianapolis, Ind., USA). All others chemicals were purchased from Becton Dickinson (Becton Dickinson Biosciences, Le Pont De Claix, France).

### Cell Lines

The human cell lines KB (epidermoid carcinoma) and HepG2 (hepatocarcinoma) were obtained from ECACC (Salisbury, UK) and grown in D-MEM medium supplemented with 10% fetal calf serum, in the presence of penicilline, streptomycine and fungizone in 75 cm² flask under 5% CO2.

The human cell lines HCT116 (colon adenocarcinoma), PC3 (prostate adenocarcinoma) and HCT15 (colon adenocarcinoma) were obtained from ECACC (Wiltshire, UK).

The human cell lines HL60 (promyeocytic leukaemia), MDA435 (breast ductal carcinoma; ATCC reference HBT129) and MDA231 (breast adenocarcinoma; ATCC reference HBT26) were obtained from ATCC. MCF7 (breast adenocarcinoma) were obtained from Dr Kassack from the University of Bonn in Germany. SK-OV3 cells (or OV3, ATCC reference HTB77) were kindly provided by NCI (Frederick, Md.).

The cell lines HCT116, HCT15, MCF7, MDA435, MDA231, PC3, OV3, HL60R and HL60 were grown in RPMI medium, in particular supplemented with 10% fetal calf serum, in the presence of penicillin, streptomycin and fungizone in 75 cm² flask under 5% CO2. Resistant HL60 cells were obtained by prolonged treatment with doxorubicin (50 μg/L medium). Resistant MCF7 (MCF7R) cells were obtained by prolonged treatment with doxorubicin. H9c2 cells (rat heart myoblast) were obtained from ATCC and grown in D-MEM medium supplemented with 10% foetal calf serum at 37° C. in 5% CO₂. The medium was changed every 2-3 days.

### Method for Measuring Inhibition of Cell Proliferation: MTS Assay

Cells were plated in 96-well tissue culture plates in 200 μl medium and cultured during 24 h before to be treated with compounds dissolved in DMSO (compound concentrations at a range of 0.5 nM to 10 μM) using a Biomek 2000 or 3000 (Beckman). Controls received the same volume of DMSO (1% final volume). After a 72 h exposure, MTS reagent (Promega) was added and incubated for 3 h at 37° C. The absorbance was then monitored at 490 nm and results were expressed as the value [1-(OD490 treated/OD490 control)×100] corresponding to the percentage of inhibition of cell proliferation. For IC50 determinations (50% of inhibition of cell proliferation), experiments were performed in separate duplicate.

### Method for Measuring Cardiotoxicity

H9c2 cells were plated for 24 h in 100 mm Petri dishes at 0,7×10⁴ cells/cm². Next, the cells were washed and cultured for 12 h in glucose-free medium (Gibco, DMEM w-L-glutamine, w/o D-glucose, sodium pyruvate) supplemented with only 1% fetal calf serum. Cells were then treated with flavaglines derivatives or their vehicle for 12 h and then were treated with doxorubicin for an additional 14 h. Kribb3 (1 μM) was preincubated for 1 h before flavaglines and doxorubicin treatment in cardiomyocytes. Cells were washed, and subsequently FACS analyses were performed.

Apoptosis was analysed by fluorescence-activated cell sorting analysis (FACS-Calibur, Becton-Dickinson Biosciences). 0,5×10⁶ cells were harvested, washed with Annexin Binding Buffer (0,01 M HEPES, 0,14 M NaCl, 2,5 mM CaCl₂) and labeled with annexin V (dilution 1:50) and propidium iodide (6,7 μg/ml). All assays were performed at least in triplicate, and the results were analyzed by BD Cell Quest Pro software (Becton-Dickinson Biosciences, Le Pont De Claix, France).

Western Blotting—H9C2 cells were plated for 24 h on 60 mm petrish dishes at 1,5×10⁴ cells/cm². Next, cells were washed and cultured for 12 h in glucose-free medium (Gibco, DMEM w-L-glutamine, w/o D-glucose, sodium pyruvate) supplemented with only 1% fetal calf serum. Cells were then incubated with FL3 or vehicle for 1 h or 4 h and after appropriate treatment washed twice with Phosphate-Buffered Saline (PBS). Cells were harvested with lysis buffer (50 mM Tris-Hcl, 1 mM EDTA, 100 mM NaCl, 0,1% SDS, 1% NP-40, 1 mM Na₃VO₄, 1 ug/mL aprotinin, 1 ug/mL pepstatin, 1 ug/mL leupeptin, pH=7). Whole cell lysates were centrifugated at 12000×g for 15 min at 4° C. The supernatant (20 ug) were mixed with a loading buffer, denatured at 100° C. for 3 min, electrophoresed on 12% SDS-PAGE under denaturing conditions, and transferred to a polyvinylidene difluoride membrane. Blots were then incubated with a blocking solution containing 5% fat-free milk powder in PBS plus Tween (0.5% Tween 20) (PBS-T) at room temperature for 1 h. After three washes with PBS-T for 10-min intervals, blots were incubated overnight at 4° C. under gentle shacking with respective primary antibody (rabbit polyclonal anti-phospho-HSP27 (1:500); rabbit polyclonal anti-HSP27 (1:500)) diluted in PBS-T containing 0,5% fat-free milk powder. After three washes with PBS-T, membrane was incubated for 1 h at room temperature under gentle shacking with a horseradish peroxidase-conjugated secondary goat anti-rabbit IgG (1:1,000 dilution) in PBS-T containing 0,5% fat-free milk powder. The expected bands were visualized after incubation by enzyme-linked chemiluminescence for 5 min and quantified by scanning laser densitometry, normalizing to total amounts of the corresponding proteins.

### Effect of Rocaglaol (FL1) and its Derivatives (FL2, FL,3 and FL4) on Human Cancer Cell Viability

The effect of rocaglaol and its derivatives was determined on human cancer cell lines by MTS assay after a 72 h treatment. As shown in table 1, FL1 (racemic rocaglaol) reduced the cell proliferation and viability more effectively than the anticancer drug doxorubicin on a panel of human cancer cell lines. Effects were observed in the low nanomolar range. Removing the methoxy group present on ring A decreased potency more than 1 000 times (compound FL2). Conversely, the replacement of the methoxy group on ring A by a bromine atom improved the cytotoxicity on all these cancer cell lines (compound FL3). The rank of activity (H<<MeO<Br) suggests a preference for an hydrophobic substituent in the para position of ring A. On contrary, introducing a methoxy on the para position of ring B was detrimental for the cytotoxicity (FL4).

TABLE 1
Inhibition of cell proliferation by flavaglines FL1 to FL4
on various human cancer cell lines (IC₅₀, nM).

| Cell lines | FL1 | FL2 | FL3 | FL4 | Doxorubicin |
|---|---|---|---|---|---|
| KB | 2 | 2500 | <1 | >10000 | 1 |
| MCF7 | 4 | 10000 | 1 | NI | 12 |
| MCF7R | 4 | 10000 | <1 | >10000 | 58 |
| HCT116 | 6 | 6000 | <1 | >10000 | 6 |
| HCT15 | 6 | 8000 | 1 | NI | 81 |
| HepG2 | 70 | >10000 | 4 | NI | 240 |
| HL60 | 3 | 6000 | <1 | >10000 | 13 |

In addition to FL3 compound, the in vitro cytotoxicity of the new synthesized flavaglines, FL5-16 and FL18 was also evaluated on a variety of human cancer cell lines from nasopharynx (KB), neutrophil (HL60 and HL60R), colon (HCT116), breast (MDA435 and MDA231), ovary (OV3), and prostate (PC3)-derived neoplasms by the MTS assay. Results are summarized in Table 2 with the values of taxotere, doxorubicine and vinblastine shown for comparison.

TABLE 2
Cytotoxicity of flavaglines analogs against human cancer cell lines (IC₅₀, nM)

| Compound | KB | HL60 | HL60R | HCT116 | MDA435 | MDA231 | OV3 | PC3 |
|---|---|---|---|---|---|---|---|---|
| FL3 | 15 | 5.5 | 4.5 | 7.6 | 8.7 | 3.8 | 8.5 | 17 |
| FL5 | 165 | 65 | 44 | 96 | 65 | 27 | 101 | 174 |
| FL6 | 255 | 115 | 67 | 108 | 205 | 48 | 251 | 224 |
| FL7 | 340 | 195 | 121 | 201 | 298 | 51 | 259 | 273 |
| FL8 | 35 | 15 | 28 | 13 | 13.4 | 5.7 | 37 | 33 |
| FL9 | 22 | 16 | 78 | 15 | 16.3 | 10 | 29 | 31 |
| FL10 | 24 | 14 | 14 | 18 | 15 | 8.7 | 21 | 29 |
| FL11 | 1 710 | 1 120 | 1280 | 1420 | 1110 | 1070 | 1460 | 2 520 |
| FL12 | 7 | 4 | 12 | 5.9 | 3.2 | 2.7 | 6.7 | 7.2 |
| FL13 | 4 | 2 | 16 | 3.8 | 2.6 | 1.9 | 3.8 | 3.3 |
| FL14 | 2 | 1 | 18 | 1.8 | 1.5 | 1.0 | 2.1 | 3.7 |
| FL15 | 60 | | | | | | | |
| FL16 | 28 | | | | | | | |
| FL18 | 14 | | | | | | | |
| FL19 | | 7 | | | | | | |
| FL20 | | 40 | | | | | | |
| FL21 | | 50 | | | | | | |
| FL22 | | 44 | | | | | | |
| FL23 | | 2 | | | | | | |
| FL24 | | 580 | | | | | | |
| FL25 | | 16 | | | | | | |
| Doxorubicin | 40 | 30 | 990 | 22 | 3.5 | 0.047 | 41 | 186 |
| Taxotere | 0.17 | 0.5 | 531 | 0.52 | 0.25 | 0.018 | 0.51 | 1.4 |
| Vinblastin | 0.8 | 1.11 | >100 | 1.42 | 1.24 | 0.63 | 2.7 | 5.5 |

The inventors observed that FL3 maintained its cytotoxicity on HL60 and MCF7 cell lines, even when they became resistant to doxorubicin.

The inventors started their structure-activity relationship investigation by introducing an ester or amide moiety on position 2 to FL3. Methyl ester FL10 exhibited a slightly reduced cytotoxicity than did compound FL3. On the opposite, introduction of a tertiary amide (FL12) significantly enhanced cytotoxicity. This effect was more pronounced with secondary amide FL13 and primary amide FL14 on most cell lines. However both FL13 and FL14 were less active that reference compound FL3 on HL60R cells, which have developed resistance to chemotherapy by overexpressing the P-glycoprotein (P-gp), a plasma membrane protein encoded by the multidrug resistance (MDR1) gene.

Next, the inventors examined the requirement of the 8-methoxy group for the cytotoxicity on cancers cells. 8-Demethoxy compounds FL6, FL5, FL8 and FL9 were significantly less active (ED50 4 to 20 times higher) than cognate compounds FL3, FL10, FL12 and FL13, indicating a preference but not an absolute requirement of a methoxy group in position 8 for cytotoxicity. In this 8-demethoxy series, the introduction of an amide at C-2 diminished also the cytotoxicity on HL60R.

Replacement of the bromine in 4′-position of FL10 by a methoxy (FL7) was detrimental to the cytotoxicity activity, as it was previously observed with FL3 and rocaglaol.

Rocaglaol (FL1) and Rocaglaol Derivatives (FL2, FL3 and FL4) do not have any Deletetious Effect on the Doxorubicin Cytotoxicity

To test the cytotoxic effect of combination of FL1 or FL3 with doxorubicin, the inventors selected HepG2 cells of hepatocellular carcinoma which display a low sensitivity toward clinically used anticancer drugs.

The inhibition of HepG2 cell proliferation with FL1, FL3 or doxorubicin alone and with doxorubicin associated with FL1 or FL3 is shown FIG. 7. These results demonstrated that rocaglaol or rocaglaol derivatives do not have any deleterious effect on doxorubicin cytotoxicity, on contrary, they strengthen this activity on cancer cells.

### Effect of Rocaglaol and its Derivatives on Doxorubicin Cardiotoxicity

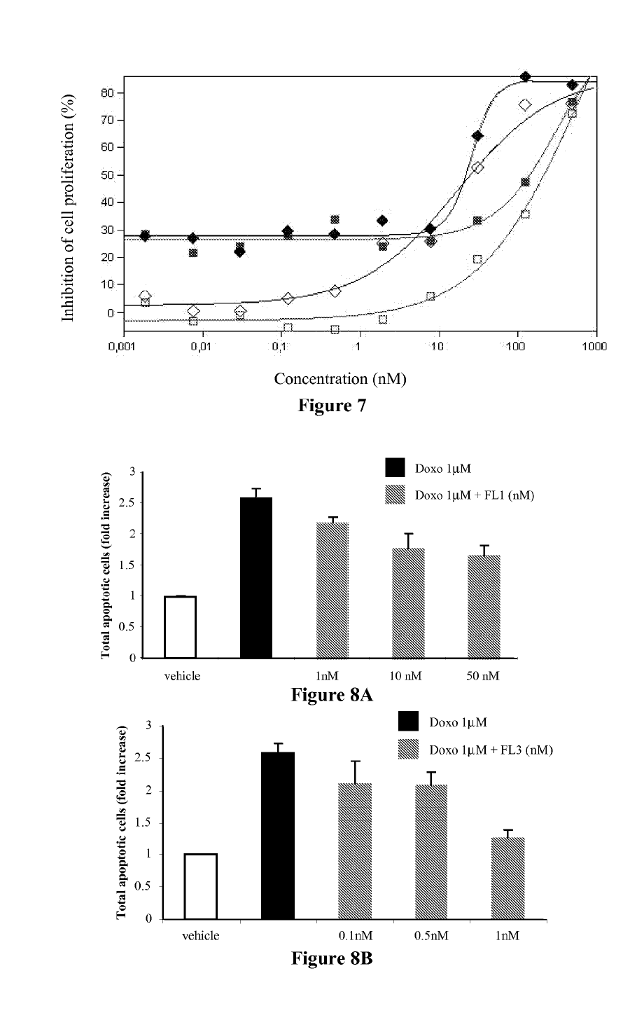

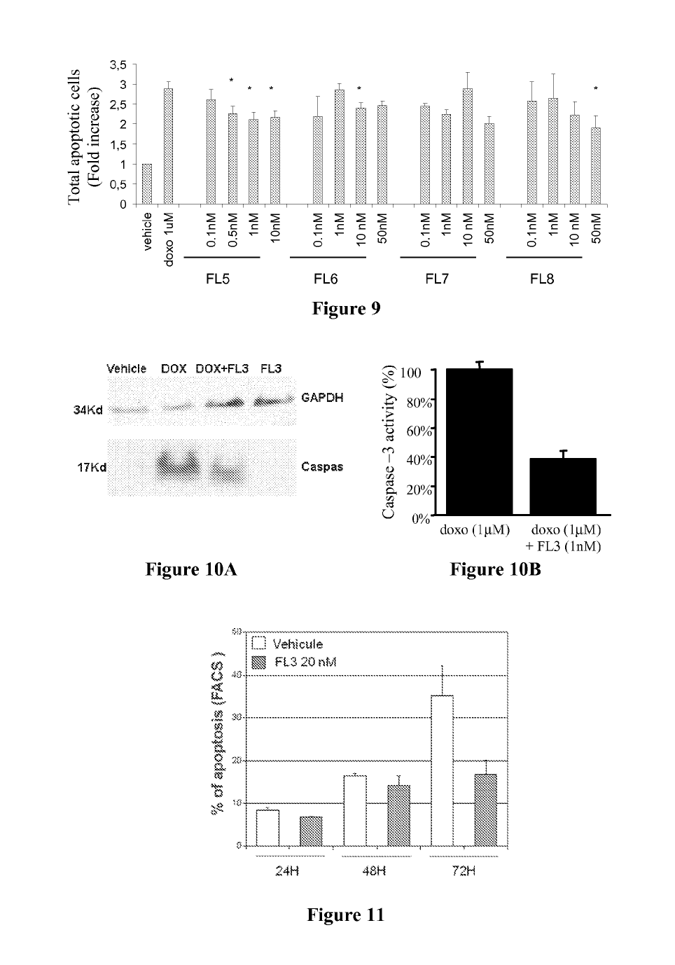

Apoptosis was induced in H9c2 cardiomyocytes by doxorubicin and detected by FACS analysis after labeling the cells with annexin and PI. These cardiomyoblasts derived from rat heart represent an established model of doxorubicin cardiotoxicity in vitro. Incubation of the cells with 1 μM doxorubicin for 14 h induced 32% apoptosis (total apoptotic cells). It was observed that pre-incubation of H9c2 cells with different concentrations of rocaglaol and its derivatives significantly reduced the apoptosis induced by doxorubicin (FIGS. 8 and 9).

The most active compound, FL3, inhibited doxorubicin-induced apoptosis in a concentration-dependent manner reaching its maximum at 1 nM (FIG. 8B). At this concentration, FL3 diminished by 70% the apoptosis induced by 1 μM doxorubicin (FIG. 10A). FIG. 13 shows the cardioprotective effect of FL1-10 and FL12-18 on apoptosis induced by doxorubicin in H9c2 cells at different concentrations at 10 or 100 nM of rocaglaol derivatives. Western blot analysis with antibody specific for the active form of caspase 3 (Chemicon International, ref AB3623) revealed that FL3 reduced doxorubicin-mediated caspase-3 activity. Indeed, H9c2 cardiomyocytes incubated for 14 h with 1 μM doxorubicin plus 1 nM FL3 failed to activate the cleavage of pro-caspase 3 in caspase 3 (FIG. 10B). FL3 may induce the death of cancer cells via activation of the AIF and caspase-12 pathways (Thuaud et al, 2009, J. Med. Chem., 52, 5176-5187). Therefore, it offers important advantages as antineoplastic agents since it acts synergistically with anthracyclines independently of caspases-3, -7, -8 and -9, suggesting that it would retain its activity in cells refractory to activation of these caspases. Considering that drug resistance and side effects are the two major obstacles limiting the efficacy of cancer chemotherapy, rocaglaol derivatives of the invention hold substantial potential for the treatment of cancers.

### FL3 Protects Cardiomyocytes Against Serum Starvation

To further explore the scope of flavagline cardioprotection, the inventors examined whether FL3 could protect H9c2 cardiomyocytes from stress due to serum starvation (1% serum). FL3 (20 nM) strongly reduced the apoptosis induced by a 72 h serum starvation treatment (FIG. 11).

### FL3 Alleviates Doxorubicin Cardiotoxicity in Mice

The inventors explored whether FL3 displays some cardioprotection in vivo. Administration of FL3 (0.1 mg/kg i.p., 2×/day for 4 days) protected mice from the cardiotoxicity induced by doxorubicine (20 mg/kg i.p.): they observed a 70% survival rate for the mice that received FL3. The survival rate for the mouse who received the vehicle was only 30% (see Table 3). This data indicates also that FL3 displays a proper bioavailabity.

TABLE 3
in vivo cardioprotection

| Treatment | Survival rate |
|---|---|
| Doxorubicin (1 x, 20 mg/kg i.p.) | 30% |
| Doxorubicin (1 x, 20 mg/kg i.p.) + | 70% |
| FL3 (8 x, 0.1 mg/kg i.p.) | |
| FL3 (8 x, 0.1 mg/kg i.p.) | 100% |

### FL3 Cardioprotection is Mediated Through HSP27 Phosphorylation

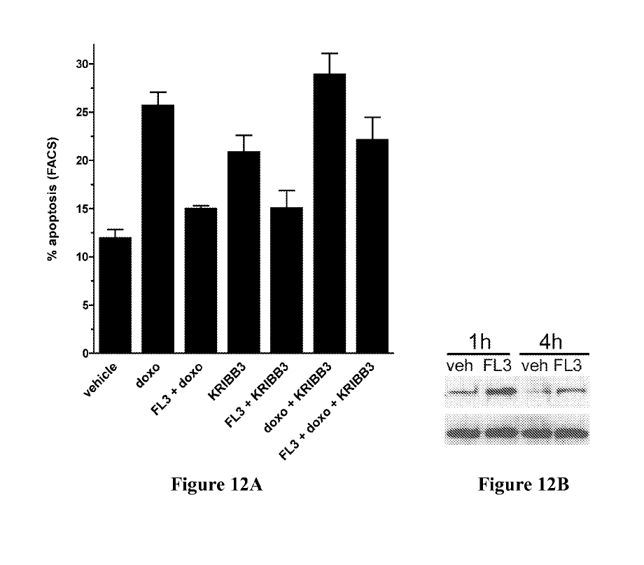

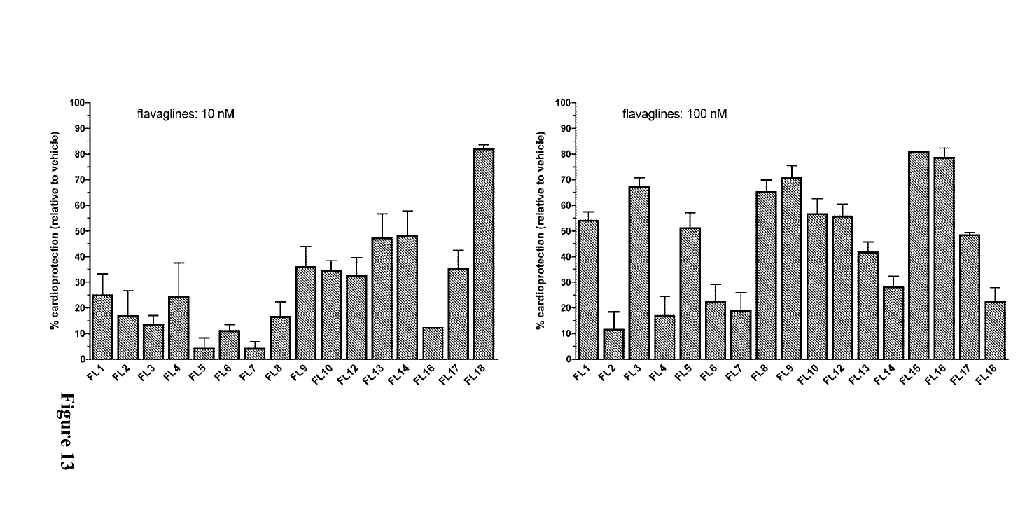

Phosphorylation of the small heat shock protein HSP27 (or its murine homolog HSP25) induces a cascade of events that protect cardiomyocytes from many stresses, including doxorubicin cardiotoxicity. To examine whether this protein is involved in FL3 cardioprotection, the inventors tested whether this effect could be blocked by KRIBB3, a synthetic molecule that inhibits HSP25/27 phosphorylation. Interestingly, a 1 h treatment of H9c2 cardiomyocytes with KRIBB3 (1 μM) induced a cytotoxicity that was significantly alleviated by a co-treatment with FL3 (100 nM), indicating that FL3 effects are opposed to those of KRIBB3. As expected KRIBB3 worsened doxorubicin cytotoxicity, but again, this effect was reversed by FL3: cells treated with doxorubicin, KRIBB3 and FL3 all together displayed the same level of apoptosis as those treated with doxorubicin alone. These results prompted the inventors to examine whether FL3 induces HSP27 phosphorylation (FIG. 12B). Western blot analysis showed indeed a high level of phosphorylation after a 1 h treatment, which significantly decreased after 4 h.

### CONCLUSION

These experiments demonstrated that rocaglaol and its derivatives according to the present invention are able to prevent or limit cardiomyocyte apoptosis, in particular doxorubicin-induced apoptosis.

Indeed, the inventors have shown that rocaglaol and its derivatives protect H9c2 cardiomyocytes from apoptosis induced by doxorubicin and serum starvation. Interestingly these two stresses are of different nature: doxorubicin induces an oxidative stress, while serum starvation blocks growth factors signaling that is necessary to cell survival. Anthracyclines such as doxorubicin, epirubicin, and daunorubicin are among the most anticancer drugs used in chemotherapy, in spite of their acute and chronic cardiotoxicities, which are dose related, cumulative and irreversible. Eventually, these myocardial dysfunctions can lead to congestive heart failure. Fortunately, the anticancer effects and the cardiotoxicity are not mediated by the same mechanisms. The former is due to an inhibition of topoisomerase II, while the latter is caused by reactive reactive oxygen species (ROS) that are generated by a redox cycling that involves the reversible reduction of the anthracycline by NADPH and a reoxidation by oxygen. The iron chelator dexrazoxane is the only cardioprotective agents with proven efficacy in cancer patients receiving anthracycline chemotherapy. However this efficacy is limited and effects on survival failed to be established.

Interestingly, the relative order of activity (FL2≈FL4<<FL1<FL3) for cardioprotection were similar to those for cytotoxicity for cancer cells, suggesting that these compounds probably act through the same molecular target. Remarkably, 50 nM FL3 alleviated by 61% the apoptosis induced by 20 times superior concentrations of doxorubicin. This effect was accompanied by a concomitant suppression of the induction of caspase-3 by doxorubicine. Same tendencies have been observed for the other rocaglaol derivatives. Indeed, FL5, FL8-10 and FL12-18 showed activity for cytotoxicity and cardioprotection, whereas FL6, FL7 and FL11 showed no or weak such activity (FIG. 13).

The inventors showed that this cardioprotection is mediated by HSP27 phosphorylation. The search for cardioprotective agents that alleviate anthracyclines cardiotoxicity in the last 30 years has led to drugs with limited, if any, efficacy in clinic. Considering that anthracyclines are the most widely used anticancer drug, the discovery of a new class of cardioprotective agents is of paramount importance in clinic. The benefit of an association of rocaglaol derivatives to anthracyclines might be triple: flavaglines could enhance the tumoricide effects of anthracyclines, delay chemoresistance evolution and alleviate the main adverse effects of anthracyclines, cardiotoxicity. A drug with all these attributes would greatly improve current treatments.

### REFERENCES

Berge, et al. J. Pharmaceutical Sciences, 1977, 66: 1-19.
Bueno O. F., et al. Circ Res. 2004; 94: 91-9.
Diedrichs N., et al. Eur J Org Chem. 2005, 9, 1731-1735.
Dobler M. R., et al. Tetrahedron Lett. 2001; 42: 8281-8284.
Fahrig T., et al. Mol Pharm. 2005; 67: 1544-1555.
Gerard B., et al. J Am Chem Soc 2004; 27: 126: 13620-1.
Hausott B, et al. Int J Cancer. 2004; 109:933-940.
Ito T, et al. J Biol Chem. 2007; 282: 1152-60.
Karmazyn M., et al. Can. J. Physiol. 1991, 69:719-730.
Kim S, et al. Anticancer Res 2007; 27:2175-83.
Kim S, et al. Anticancer Agents Med Chem 2006; 6:319-45.
Mi Q, et al. Anticancer Research 2006, 26:947-952.
Pai V. B., et al. Drug Saf. 2000; 22:263-302.
Proksch P, et al. Curr Org Chem 2001; 5:923-38.
Steinherz L. J., et al. Med. Pediatr. Oncol. 1995; 24:352-361.
Swain S. M., et al. Cancer. 2003; 97:2869-2879.
Van Dalen E. C., et al. Cochrane Database Syst Rev. 2005 Jan. 25; (1):CD003917.
Van Dalen E. C., et al. Cochrane Database Syst Rev. 2008: CD003917.
Wouters, K. A. et al. Br J Haematol. 2005; 131:561-78.

1-34. (canceled)

35. A method for preventing or limiting the cardiotoxicity of an antineoplastic agent in a subject comprising administering to said subject a therapeutically effective amount of a rocaglaol derivative of the formula (II) 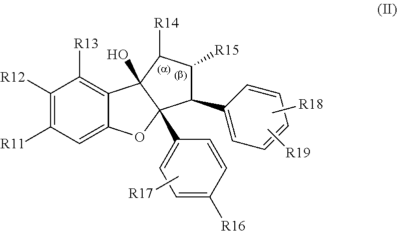
wherein
R¹¹ is alkoxy, optionally substituted;
R¹² is hydrogen;
or R¹¹ and R¹² together form a —O—CH₂—O-unit;
R¹³ is selected from the group consisting of hydrogen and (C₁-C₃)-alkoxy;
R¹⁴ is selected from the group consisting of hydroxyl, oxo group, and —OCOR²⁰, R²⁰ being selected from the group consisting of hydrogen and (C₁-C₃)-alkyl, ═N—OR²⁶ with R²⁶ being H or methyl, —NH—(CH₂)ₘ—R²⁷, wherein m is 0 or 1 and R²⁷ is selected from the group consisting of H, —OH, —SO₂Me, and —COR²⁸ with R²⁸ being H or (C₁-C₃)-alkyl;
R¹⁵ is selected from the group consisting of hydrogen, —COOR²¹, —CONR²²R²³ and —CONH(CH₂)ₙOH, wherein n is 2, 3 or 4, wherein
R²¹ and R²³ are independently selected from the group consisting of hydrogen and (C₁-C₃)-alkyl and R²² is selected from the group consisting of (C₁-C₃)-alkoxy, (C₁-C₃)-alkyl and hydrogen, or R²² and R²³ together form a 5 atom heterocycle, optionally substituted by a group NHCO—(C₁-C₄)-alkyl;
or R¹⁴, R¹⁵ and carbons (α) and (β) bearing R¹⁴ and R¹⁵ together form 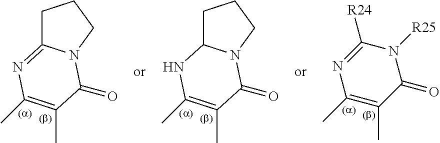
wherein R²⁴ and R²⁵ are independently selected from the group consisting of (C₁-C₃)-alkyl and hydrogen;
R¹⁶ is selected from the group consisting of hydrogen, (C₁-C₃)-alkoxy and halogen;
R¹⁷ is in ortho-position to R₁₆ and is selected from the group consisting of hydrogen, (C₁-C₃)-alkoxy and hydroxyl or form together with R¹⁷ a —O—CH₂—O-unit; and
R¹⁸ and R¹⁹ are independently selected from the group consisting of hydrogen and halogen;
or any pharmaceutically acceptable salt thereof.

36. The method according to claim 35, wherein the rocaglaol derivative of the formula (II) has one or several of the following features:
a) R¹² is H and R¹¹ is selected from the group consisting of methoxy and ethoxy;
b) R¹³ is selected from the group consisting of hydrogen, methoxy and ethoxy;
c) R¹⁴ is selected from the group consisting of hydroxyl and —OCOR²⁰, R²⁰ being selected from the group consisting of hydrogen, methyl and ethyl, —NH—(CH₂)ₘ—R²⁷, wherein m is 0 and R²⁷ is —SO₂Me or —COR²⁸ with R²⁸ being H or (C₁-C₃)-alkyl;
d) R¹⁵ is selected from the group consisting of hydrogen, —COOR²¹, and —CONR²²R²³, wherein R²¹ and R²³ are independently selected from the group consisting of hydrogen and methyl, and R²² is selected from the group consisting of hydrogen, methyl and methoxy;
e) R¹⁶ is selected from the group consisting of hydrogen, (C₁-C₃)-alkoxy and halogen;
f) R¹⁷ is hydrogen; and
g) R¹⁸ is in para and is H or fluorine and R¹⁹ is hydrogen.

37. The method according to claim 35, wherein the rocaglaol derivative presents the formula (IIa) or (IIb) 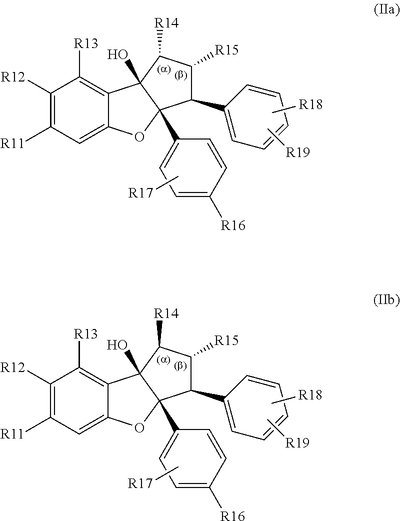
wherein the definition of R11, R12, R13, R14, R15, R16, R17, R18 and R19 is the same as in claim 35.

38. The method according to claim 35, wherein the rocaglaol derivative has the formula (IIa) and R¹⁴ is selected from the group consisting of hydroxyl and —OCOR²⁰, R²⁰ being selected from the group consisting of hydrogen and hydrogen, methyl and ethyl, and ═N—OR²⁶ with R²⁶ being H or methyl.

39. The method according to claim 37, wherein the rocaglaol derivative has the formula (IIb) and R¹⁴ is —NH—(CH₂)ₘ—R²⁷, wherein m is 0 and R²⁷ is —SO₂Me or —COR²⁸ with R²⁸ being H or (C₁-C₃)-alkyl.

40. The method according to claim 35, wherein the rocaglaol derivative is selected from the group consisting of FL1, FL3, FL5, FL6, FL7, FL8, FL9, FL10, FL12, FL13, FL14, FL15, FL16, FL17, FL18, FL19, FL20, FL21, FL22, FL23, FL24 and FL25.

41. A rocaglaol derivative of the formula (I) 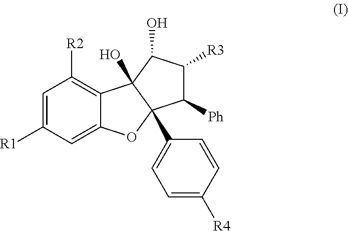
wherein
R³ is —CONH₂;
and wherein
R¹ is alkoxy, optionally substituted;
R² is selected from the group consisting of hydrogen and (C₁-C₃)-alkoxy; and
R⁴ is halogen,
or,
R¹ is alkoxy, optionally substituted;
R² is hydrogen; and
R⁴ is selected from the group consisting of hydrogen and alkoxy,
or,
R¹ is a substituted alkoxy;
R² is (C₁-C₃)-alkoxy; and
R⁴ is selected from the group consisting of hydrogen and alkoxy,
or wherein
R³ is —COOR⁵ wherein R⁵ is (C₁-C₃)-alkyl;
R¹ is alkoxy, optionally substituted;
R² is hydrogen; and
R⁴ is selected from the group consisting of hydrogen, methoxy and halogen;
or wherein
R¹ is methoxy, and R⁴ is bromine,
and wherein
R² is (C₁-C₃)-alkoxy, and
R³ is hydrogen,
or
R² is hydrogen, and
R³ is selected from the group consisting of hydrogen, —COOR⁵ and —CONR⁶R⁷, wherein
R⁵ is (C₁-C₃)-alkyl and R⁶ and R⁷ are independently selected from the group consisting of hydrogen and (C₁-C₃)-alkyl;
or any pharmaceutically acceptable salt thereof.

42. The rocaglaol derivative according to claim 41, wherein the rocaglaol derivative is selected from the group consisting of FL3, FL5, FL6, FL7, FL5, FL9 and FL14.

43. A rocaglaol derivative of the formula (II) 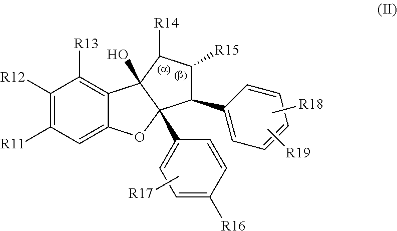
wherein
R¹¹ is alkoxy, optionally substituted;
R¹² is hydrogen;
or R¹¹ and R¹² together form a —O—CH₂—O-unit;
R¹³ is selected from the group consisting of hydrogen and (C₁-C₃)-alkoxy;
R¹⁴ is —NH—R²⁷, wherein R²⁷ is selected from the group consisting of —SO₂Me and —COR²⁸ with R²⁸ being H or (C₁-C₃)-alkyl;
R¹⁵ is selected from the group consisting of hydrogen, —COOR²¹, —CONR²²R²³ and —CONH(CH₂)ₙOH, wherein n is 2, 3 or 4, wherein
R²¹ and R²³ are independently selected from the group consisting of hydrogen and (C₁-C₃)-alkyl and R²² is selected from the group consisting of (C₁-C₃)-alkoxy, (C₁-C₃)-alkyl and hydrogen, or
R²² and R²³ together form a 5 atom heterocycle, optionally substituted by a group NHCO—(C₁-C₄)-alkyl;
R¹⁶ is selected from the group consisting of hydrogen, (C₁-C₃)-alkoxy and halogen;
R¹⁷ is in ortho-position to R₁₆ and is selected from the group consisting of hydrogen, (C₁-C₃)-alkoxy and hydroxyl or form together with R¹⁷ a —O—CH₂—O-unit; and
R¹⁸ and R¹⁹ are independently selected from the group consisting of hydrogen and halogen;
or any pharmaceutically acceptable salt thereof.

44. The rocaglaol derivative according to claim 43, wherein the rocaglaol derivative of the formula (II) has one or several of the following features:
a) R¹¹ is alkoxy and R¹² is hydrogen;
b) R¹³ is selected from the group consisting of hydrogen and methoxy;
c) R¹⁴ is —NH—R²⁷, wherein R²⁷ is selected from the group consisting of —SO₂Me and —COR²⁸ with R²⁸ being H, methyl or ethyl;
d) R¹⁵ is selected from the group consisting of hydrogen, —COOR²¹, and —CONR²²R²³, wherein R²¹ and R²³ are independently selected from the group consisting of hydrogen and methyl, and R²² is selected from the group consisting of hydrogen, methyl and methoxy;
e) R¹⁶ is selected from the group consisting of hydrogen, (C₁-C₃)-alkoxy and halogen;
f) R¹⁷ is hydrogen; and
g) R¹⁸ is in para and is H or fluorine and R¹⁹ is hydrogen.

45. The rocaglaol derivative according to claim 43, wherein the rocaglaol derivative presents the formula (IIa) or (IIb) 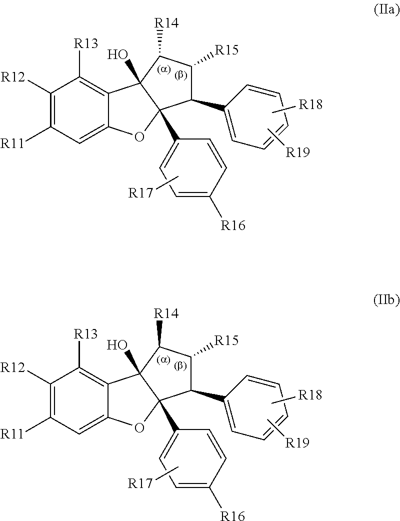
wherein the definition of R11, R12, R13, R14, R15, R16, R17, R18 and R19 is the same as in claim 43.

46. The rocaglaol derivative according to claim 45, wherein the rocaglaol derivative has the formula (IIb) and R¹⁴ is —NH—R²⁷, wherein R²⁷ is —SO₂Me or —COR²⁸ with R²⁸ being H or (C₁-C₃)-alkyl.

47. The rocaglaol derivative according to claim 43, wherein the rocaglaol derivative is selected from the group consisting of FL22, FL23, FL24 and FL25.

48. A rocaglaol derivative of the formula (IIa) 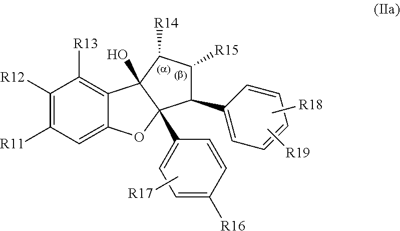
wherein
R¹² is H and R¹¹ is selected from the group consisting of methoxy and ethoxy;
R¹³ is selected from the group consisting of hydrogen, methoxy and ethoxy;
R¹⁴ is —OCOR²⁰, R²⁰ being selected from the group consisting of hydrogen, methyl and ethyl;
R¹⁵ is selected from the group consisting of hydrogen, —COOR²¹, and —CONR²²R²³, wherein R²¹ and R²³ are independently selected from the group consisting of hydrogen and methyl, and R²² is selected from the group consisting of hydrogen, methyl and methoxy; R¹⁶ is selected from the group consisting of chlorine, bromine and fluorine;
R¹⁷ is hydrogen; and
R¹⁸ is in para and is H or fluorine and R¹⁹ is hydrogen;
or any pharmaceutically acceptable salt thereof.

49. The rocaglaol derivative according to claim 48, wherein the rocaglaol derivative is selected from the group consisting of FL19, FL20 and FL21.

50. A rocaglaol derivative of the formula (IIa) 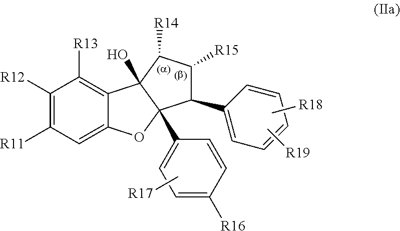
wherein
R¹² is H and R¹¹ is selected from the group consisting of methoxy and ethoxy;
R¹³ is selected from the group consisting of hydrogen, methoxy and ethoxy;
R¹⁴ is hydroxyl;
R¹⁸ is in para and is fluorine and R¹⁹ is hydrogen,
and wherein
R¹⁵ is selected from the group consisting of hydrogen, —COOR²¹, and —CONR²²R²³, wherein
R²¹ and R²³ are independently selected from the group consisting of hydrogen and methyl, and R²² is selected from the group consisting of hydrogen, methyl and methoxy; and
R¹⁶ is bromine and R¹⁷ is hydrogen;
or
R¹⁵ is selected from the group consisting of —COOR²¹, and —CONR²²R²³, wherein R²¹ and R²³ are independently selected from the group consisting of hydrogen and methyl, and R²² is selected from the group consisting of hydrogen, methyl and methoxy; and
R¹⁶ is methoxy and R¹⁷ is hydrogen;
or any pharmaceutically acceptable salt thereof.

51. The rocaglaol derivative according to claim 50, wherein the rocaglaol derivative is FL16, FL17 or FL18.

52. A pharmaceutical composition comprising a rocaglaol derivative according to claim 41 and a pharmaceutically acceptable carrier and/or excipient.

53. A pharmaceutical composition comprising a rocaglaol derivative according to claim 43 and a pharmaceutically acceptable carrier and/or excipient.

54. A pharmaceutical composition comprising a rocaglaol derivative according to claim 48 and a pharmaceutically acceptable carrier and/or excipient.

55. A pharmaceutical composition comprising a rocaglaol derivative according to claim 50 and a pharmaceutically acceptable carrier and/or excipient.
